(19) United States
(12) Patent Application Publication
(10) Pub. No.: US 2011/0135571 A1
(43) Pub. Date: Jun. 9, 2011

(54) HYBRID NANOPARTICLES AS ANTI-CANCER THERAPEUTIC AGENTS AND DUAL THERAPEUTIC/IMAGING CONTRAST AGENTS

(76) Inventors: Wenbin Lin, Chapel Hill, NC (US); William J. Rieter, Charleston, SC (US)

(21) Appl. No.: 12/918,748

(22) PCT Filed: Feb. 23, 2009
(86) PCT No.: PCT/US09/34867

§ 371 Date: Feb. 22, 2011

Related U.S. Application Data
(60) Provisional application No. 61/137,565, filed on Jul. 31, 2008.
(60) Provisional application No. 61/030,746, filed on Feb. 22, 2008.

(51) Int. Cl. A61K 9/14; C08F 26/10; B32B 5/00; A61K 31/79; A61K 51/00; A61K 49/00; A61B 5/055; A61K 9/127; C12N 5/00; A61P 35/00; B82Y 5/00

(52) U.S. Cl. 424/1.65; 525/370; 428/402; 424/78.24; 424/9.1; 424/9.3; 424/490; 424/497; 424/450; 435/375; 977/773; 977/906; 977/915

(57) ABSTRACT

The presently disclosed subject matter provides nanoscale coordination polymers for use as anticancer agents and as dual anticancer/imaging agents. The nanoscale coordination polymers can comprise a plurality of platinum metal complexes; nonplatinum anticancer drug bridging ligands complexed to multiple metal centers; or combinations thereof. The nanoscale coordination polymers can be targeted for delivery to cancer cells. They can also comprise stabilizing agents to allow for controlled and/or sustained release of anticancer agents in vivo.

### RELATED APPLICATIONS

The presently disclosed subject matter claims the benefit of U.S. Provisional Patent Application Ser. No. 61/030,746, filed Feb. 22, 2008, and U.S. Provisional Patent Application Ser. No. 61/137,565, filed Jul. 31, 2008, each of which is incorporated herein by reference in its entirety.

### GOVERNMENT INTEREST

This presently disclosed subject matter was made with U.S. Government support under Grant No. DMR-0605923 awarded by the National Science Foundation and Grant No. U54-CA119343 awarded by the National Cancer Institute of the U.S. National Institutes of Health. Thus, the U.S. Government has certain rights in the presently disclosed subject matter.

### TECHNICAL FIELD

The presently disclosed subject matter provides hybrid nanomaterials comprising coordination polymers prepared from metal complexes for use as anticancer agents and as combination anticancer and imaging agents.

### Abbreviations

° C.=degrees Celsius
δ=chemical shift
μg=microgram
μM=micromolar
BDC=benzene dicarboxylate
BTC=benzene tricarboxylate
CHCl₃=chloroform
CNS=central nervous system
CTAB=cetyltrimethylammonium bromide
DCP=direct current plasma
DLS=dynamic light scattering
DMSO=dimethylsulfoxide
DSCP=disuccinatocisplatin
ECL=electrochemiluminescence
EDTA=ethylene diamine tetraacetate
EPR=enhanced permeability and retention
g=gram
h=hour
HEPES=4-(2-hydroxyethyl)-1-piperazine-ethanesulfonic acid
H₂O=water
H₂O₂=hydrogen peroxide
ICP-MS=inductively coupled plasma-mass spectrometry
Ln=lanthanoid
M=molar
mL=millimeter
mmol=millimole
mol=mole
MRI=magnetic resonance imaging
MW=molecular weight
NaOH=sodium hydroxide
NCP=nanoscale coordination polymer
NH₃=ammonia
nm=nanometer
NMR=nuclear magnetic resonance
PDT=photodynamic therapy
PEG=poly(ethylene glycol)
PET=positron emission tomography
Pt=platinum
PVP=polyvinylpyrrolidone
PXRD=powder X-ray diffraction
RES=reticulo-endothelial system
RGD=arginine-glycine-aspartic acid
SEM=scanning electron microscope
SPECT=single photon emission computed tomography
Tb=terbium
TEM=transmission electron microscope
TEOS=tetraethyl orthosilicate
TGA=thermogravimetric analysis
UV=ultraviolet
W=water to surfactant molar ratio
wt %=weight percentage
XRT=X-ray radiation
Zn=zinc

### BACKGROUND

A variety of anticancer drugs are available for treating different types of cancers in the clinic. However, the therapeutic efficacy of these drugs is often limited by the inability to selectively deliver the drugs to tumors. Most of the currently available anticancer drugs are highly cytotoxic, and can kill normal cells along with cancerous cells. Thus, when high doses of drugs are used, there can be severe side effects. As a result, most of the currently used anticancer drugs have a rather limited therapeutic index. Such a limit on dosage prevents the complete eradication of cancer cells in a patient, and can lead to recurrence of the cancer in many patients. The limit in dosage can also predispose the recurring cancer to drug resistance, thus worsening the prognosis for the patient.

Accordingly, there is an ongoing need for new anticancer therapeutics that can be selectively delivered to tumors and/or which can provide improved therapeutic indices. There is also an ongoing need for anticancer agents whose delivery to tumors can be simultaneously observed via various imaging techniques or which can include a variety of anticancer agents.

### SUMMARY

The presently disclosed subject matter provides a nanoparticle comprising a coordination polymer comprising a plurality of platinum metal complexes. In some embodiments, the plurality of platinum metal complexes include but are not limited to a plurality of platinum (II) metal complexes, a plurality of platinum (IV) metal complexes, or a combination thereof. In some embodiments, one or more of the platinum metal complexes comprises:
a platinum metal atom;
at least one nonbridging ligand, wherein the at least one nonbridging ligand is bonded to the platinum metal atom through at least one coordination bond; and
at least one bridging ligand, wherein the at least one bridging ligand is bonded to the platinum metal atom through at least one coordination bond and comprises at least one linking moiety, wherein each of the at least one linking moiety is bonded to an additional metal atom via a coordination bond.

In some embodiments, each of the at least one linking moiety is independently selected from the group consisting of a carboxylate, a carboxylic acid, an amine, a hydroxyl, a thiol, a carbamate, an ester, a phosphate, a phosphonate, a carbonate, and an amide.

In some embodiments, each of the at least one bridging ligand is independently selected from the group consisting of a polymeric bridging ligand and a nonpolymeric bridging ligand. In some embodiments, each of the at least one bridging ligand is a nonpolymeric bridging ligand. In some embodiments, the bridging ligand comprises at least two carboxylate groups.

In some embodiments, at least one platinum metal complex comprises two bridging ligands, wherein each of the two bridging ligands is bonded to the platinum metal atom through one coordination bond and comprises at least one linking moiety. In some embodiments, each of the two bridging ligands is independently selected from the group consisting of 1,4-benzene dicarboxylate; 1,3,5-benzene tricarboxylate; succinate; and ethylene diamine tetraacetate.

In some embodiments, at least one platinum metal complex comprises one bridging ligand, wherein the one bridging ligand is bonded to the platinum metal atom through two coordination bonds and comprises at least two linking moieties. In some embodiments, the one bridging ligand is a bipyridine dicarboxylate. In some embodiments, the bipyridine dicarboxylate is selected from 2,2′-bipyridine-5,5′-dicarboxylate and 2,2′-bipyridine-4,4′-dicarboxylate.

In some embodiments, one of the at least one bridging ligand is a nonplatinum anticancer drug. In some embodiments, the nonplatinum anticancer drug is selected from the group consisting of methotrexate, folic acid, leucovorin, vinblastine, vincristine, melphalan, imatinib, pemetrexed, vindesine, anastrozole, doxorubicin, cytarabine, azathioprine, letrozole and carboxylates thereof.

In some embodiments, one of the at least one bridging ligand is a polymeric bridging ligand. In some embodiments, wherein the polymeric bridging ligand comprises one of the group consisting of poly(silsesquioxane), poly(siloxane), poly(acrylate) and poly(acrylamide).

In some embodiments, the additional metal atom is a platinum metal atom of a second platinum metal complex. In some embodiments, the additional metal atom is a nonplatinum metal atom selected from the group consisting of a transition metal atom, a lanthanide metal atom, and an actinide metal atom. In some embodiments, the additional metal atom is selected from the group consisting of Tb³⁺ and Zn²⁺.

In some embodiments, each of the at least one nonbridging ligands is independently selected from the group consisting of NH₃, a primary amine, a secondary amine, a diamine, an aromatic amine, a halide, and hydroxide. In some embodiments, the diamine is a cyclohexanediamine. In some embodiments, each of the at least one nonbridging ligands is independently selected from the group consisting of NH₃ and chloride.

In some embodiments, each of the plurality of platinum metal complexes is independently selected from the group consisting of:
Pt[(NH₃)₂(Cl)₂(O₂CCH₂CH₂CO₂)₂];
Pt[(NH₃)₂(Cl)₂{O₂CC₆H₃(CO₂)₂}₂];
dichloro(2,2′-bipyridine-4,4′-dicarboxylato)platinum (II);
dichloro(2,2′-bipyridine-5,5′-dicarboxylato)platinum (II); and
Pt[(NH₃)₂(Cl)₂(ethylene diamine tetraacetate)₂].

In some embodiments, the nanoparticle has a diameter ranging between about 20 nm and about 250 nm. In some embodiments, the nanoparticle has a diameter ranging between about 40 nm and about 70 nm.

In some embodiments, the nanoparticle further comprises one or more of the group consisting of a photosensitizer, a radiosensitizer, a radionuclide, a passivating agent, an imaging agent, and a targeting agent. The imaging agent can be selected from the group consisting of an optical imaging agent, a magnetic resonance imaging (MRI) agent, a positron emission tomography (PET) imaging agent, and a single photon emission computed tomography (SPECT) imaging agent. In some embodiments, the optical imaging agent is a luminescent agent.

In some embodiments, the targeting agent is selected from the group consisting of a small molecule, a peptide, and a protein. In some embodiments, the targeting agent binds to a receptor or ligand present on a cancer cell. In some embodiments, the targeting agent comprises cyclic(RGDfk).

In some embodiments, an outer surface of the nanoparticle is chemically modified with one or more of a passivating agent, a targeting agent, and an imaging agent. In some embodiments, the passivating agent comprises poly(ethylene glycol).

In some embodiments, the nanoparticle comprises a core and an outer layer, the core comprising a coordination polymer comprising a plurality of platinum metal complexes, and the outer layer surrounding the core and comprising one or more of a metal oxide, a lipid bilayer, an organic polymer, a silica-based polymer, and combinations thereof. In some embodiments, the organic polymer is polyvinylpyrolidone (PVP). In some embodiments, the outer layer is polyvinylpyrolidone (PVP), SiO₂, or a combination thereof. In some embodiments, the outer layer has a thickness ranging between about 1 nm and about 10 nm.

In some embodiments, the presently disclosed subject matter provides a pharmaceutical composition comprising a nanoparticle comprising a coordination polymer comprising a plurality of platinum metal complexes and a pharmaceutically acceptable carrier. In some embodiments, the pharmaceutical composition is pharmaceutically acceptable in humans. In some embodiments, the pharmaceutical composition comprises one of a liposome and a microemulsion.

In some embodiments, the presently disclosed subject matter provides a method of inhibiting proliferation of a cancer cell, the method comprising contacting the cancer cell with a nanoparticle, wherein the nanoparticle comprises a coordination polymer comprising a plurality of platinum metal complexes. In some embodiments, the cancer cell is selected from a skin cancer cell, a connective tissue cancer cell, an esophageal cancer cell, a head and neck cancer cell, a breast cancer cell, a lung cancer cell, a stomach cancer cell, a pancreatic cancer cell, an ovarian cancer cell, a cervical cancer cell, a uterine cancer cell, an anogenital cancer cell, a kidney cancer cell, a bladder cancer cell, a colon cancer cell, a prostate cancer cell, a retinal cancer cell, a central nervous system cancer cell, and a lymphoid cancer cell.

In some embodiments, the presently disclosed subject matter provides a method of treating cancer in a subject in need of treatment thereof, the method comprising administering to the subject a nanoparticle comprising a coordination polymer, wherein the coordination polymer comprises a plurality of platinum metal complexes. In some embodiments, the cancer is selected from a skin cancer, a connective tissue cancer, an esophageal cancer, a head and neck cancer, a breast cancer, a lung cancer, a stomach cancer, a pancreatic cancer, an ovarian cancer, a cervical cancer, a uterine cancer, an anogenital cancer, a kidney cancer, a bladder cancer, a colon cancer, a prostate cancer, a retinal cancer, a central nervous system cancer, and a lymphoid cancer. In some embodiments, the subject is a mammal.

In some embodiments, the method further comprises imaging delivery of the nanoparticle in one or more tissue or organ in the subject following administration of the nanoparticle. In some embodiments, the method further comprises administering to the subject an external stimulus selected from the group consisting of laser light and X-ray radiation.

In some embodiments, the presently disclosed subject matter provides a method of synthesizing a nanoparticle comprising a coordination polymer comprising a plurality of platinum metal complexes, wherein the method comprises precipitation or use of a microemulsion system.

In some embodiments, the method comprises providing a solution comprising a first solvent, at least one bridging ligand precursor, and a plurality of platinum diaqua complexes selected from the group consisting of platinum (II) diaqua complexes, platinum (IV) diaqua complexes, and mixtures thereof; and adding a second solvent to the solution to precipitate the nanoparticle.

In some embodiments, the method further comprises adjusting the pH of the solution prior to adding the second solvent. In some embodiments, the first solvent comprises water, dimethyl sulfoxide (DMSO), or a combination thereof. In some embodiments, the at least one bridging ligand precursor is selected from the group consisting of a benzene dicarboxylic acid, a benzene dicarboxylate, a carboxylate-substituted styrene, a carboxylate-substituted silyl ether, a bipyridine dicarboxylic acid, a bipyridine dicarboxylate, a dicarboxylic anhydride, a diacyl dichloride, and a nonplatinum anticancer drug.

In some embodiments, the second solvent is selected from the group consisting of acetone, an alcohol, ether, and acetonitrile. In some embodiments, the solution further comprises an additional component, wherein the additional component is selected from the group consisting of a radionuclide, an imaging agent, a photosensitizer, and a radiosensitizer, and adding the second solvent co-precipitates the additional component, thereby incorporating the additional component into the nanoparticle.

In some embodiments, the method comprises providing a first mixture comprising a microemulsion system comprising water, an organic solvent, a surfactant, and a co-surfactant; adding to the first mixture an aqueous solution comprising a platinum metal complex, thereby forming a second mixture, wherein the platinum metal complex comprises a platinum metal atom, one or more nonbridging ligands, and at least one ligand bound to the platinum metal atom by at least one coordination bond and comprising at least one prelinking moiety, wherein the at least one prelinking moiety is a group that can form a coordination bond with an additional metal atom; stirring the second mixture until the second mixture is visably clear; providing a third mixture comprising a microemulsion system comprising water, an organic solvent, a surfactant, and a co-surfactant; adding to the third mixture an aqueous solution comprising a nonplatinum metal compound, thereby forming a fourth mixture; stirring the fourth mixture until the fourth mixture is visably clear; adding the fourth mixture and the second mixture to form a fifth mixture; and stirring the fifth mixture for a period of time, thereby synthesizing the nanoparticle.

In some embodiments, the method comprises providing a microemulsion system comprising water; an organic solvent; a surfactant; a co-surfactant; a polymerizable monomer; and a platinum metal complex, wherein the platinum metal complex comprises a platinum metal atom, one or more nonbridging ligands, and at least one ligand bonded to the platinum metal atom by at least one coordination bond and comprising at least one prelinking moiety, where the at least one prelinking moiety is a moiety that can react with the polymerizable monomer.

In some embodiments, the prelinking moiety is selected from the group consisting of an alkyl halide, an acyl halide, a silyl ether, an alkene, an alkyne, a carboxylic acid, an amine, an ester, an anhydride, and an isocyanate. In some embodiments, the polymerizable monomer is selected from the group consisting of a silyl ether, acrylic acid, and acrylamide.

In some embodiments, the method further comprises isolating the nanoparticle via centrifugation. In some embodiments, the method further comprises coating the nanoparticle with one or more of the group consisting of a metal oxide, a lipid bilayer, an organic polymer, a silica-based polymer, and combinations thereof. In some embodiments, the method further comprises grafting onto a surface of the nanoparticle one or more of a photosensitizer, a radiosensitizer, a radionuclide, an imaging agent, a passivating agent, and a targeting agent.

In some embodiments, the presently disclosed subject matter provides a coordination polymer comprising a plurality of platinum metal complexes wherein the platinum metal complexes are linked via bridging ligands, wherein each bridging ligand is independently selected from the group consisting of a nonpolymeric bridging ligand and a polymeric bridging ligand.

In some embodiments, the presently disclosed subject matter provides a coordination polymer comprising a plurality of nonplatinum metal complexes wherein the nonplatinum metal complexes are linked via bridging ligands, wherein one or more of the bridging ligands are a nonplatinum anticancer drug.

It is an object of the presently disclosed subject matter to provide anticancer therapeutic agents and dual anticancer/imaging contrast agents.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1 is a drawing showing the structures of platinum (II) anticancer prodrugs and their active diaqua complexes.

FIG. 2 is a schematic drawing showing the synthesis of a platinum (II) metal complex-based coordination polymer.

FIG. 3 is a schematic drawing showing the synthesis of a platinum (IV) metal complex-based coordination polymer.

FIG. 4 is a schematic drawing showing the synthesis of a coordination polymer comprising both platinum (II) and platinum (IV) metal complexes.

FIG. 5 is a series of drawings showing the structures of exemplary small molecule, organic anticancer drugs that can be used as bridging ligands in coordination polymer nanoparticles.

FIG. 6 is a series of three schematic diagrams showing the synthesis of nanoparticles comprising coordination polymers comprising platinum metal complexes having polymeric bridging ligands.

FIG. 7 are two drawings showing the structures of exemplary photo- and radiosensitizers that can be incorporated into coordination polymer nanoparticles.

FIG. 8 is a schematic diagram showing the synthesis of a silica layer stabilized, c(RGDfK)-targeted, bimetallic coordination polymer nanoparticle.

FIG. 9 are scanning electron micrograph (SEM) images of cis-Pt(NH₃)₂(benzene dicarboxylate) nanoclusters synthesized via the rapid addition of acetone to a precursor (aqueous) solution. Scale markings in the left-hand image are every 5 microns. Scale markings in the right-hand image are every 1 micron.

FIG. 10 are scanning electron micrograph (SEM) images of cis-Pt(NH₃)₂(benzene dicarboxylate) nanoclusters synthesized via the rapid addition of an acetone/ethanol mixture (1:1 v/v) to a precursor (aqueous) solution. Scale markings in the left-hand image are every 10 microns. Scale markings in the right-hand image are every 3 microns.

FIG. 11 are scanning electron micrograph (SEM) images of Tbₓ[c,c,t-Pt(NH₃)₂(Cl)₂(O₂CCH₂CH₂CO₂)₂]_{y} nanoparticles synthesized via cationic microemulsions with [water]/[surfactant] ratios of 15 (left image) and 20 (right image). Scale markings in both images are every 5 nm.

FIG. 12 are transmission electron micrograph (TEM) images of Tbₓ[c,c,t-Pt(NH₃)₂(Cl)₂(O₂CCH₂CH₂CO₂)₂]_{y} nanoparticles (NCP-1) synthesized via the rapid addition of an methanol to a precursor (aqueous) solution. The scale marking in the left-hand corner of the left-hand image represents 0.2 microns. The scale marking in the left-hand corner of the right-hand image represents 50 nm.

FIG. 13 are scanning electron micrograph (SEM) images of Tbₓ[c,c,t-Pt(NH₃)₂(Cl)₂(O₂CCH₂CH₂CO₂)₂]_{y} nanoparticles synthesized via the rapid addition of an ethanol/methanol mixture (1:1 v/v) to a precursor (aqueous) solution.

FIG. 14 are scanning electron micrograph (SEM) images of Znₓ[c,c,t-Pt(NH₃)₂(Cl)₂(O₂CCH₂CH₂CO₂)₂]_{y} nanoparticles synthesized via the rapid addition of methanol to a precursor (aqueous) solution.

FIG. 15 are scanning electron micrograph (SEM) images of poly(vinylpyrrolidone) (PVP) coated Tbₓ[c,c,t-Pt(NH₃)₂(Cl)₂(O₂CCH₂CH₂CO₂)₂]_{y} nanoparticles (NCP-1/PVP). The scale marking in the left-hand corner of the left-hand image represents 0.2 microns. The scale bar in the left-hand corner of the right-hand image represents 50 nm.

FIG. 16A is a transmission electron micrograph (TEM) image of silica-coated Tbₓ[c,c,t-Pt(NH₃)₂(Cl)₂(O₂CCH₂CH₂CO₂)₂]_{y} nanoparticles (i.e., NCP-1′) isolated after 2 hours of exposure to a silica-coating solution. The scale bar in the left-hand corner of the image represents 50 nm.

FIG. 16B is a transmission electron micrograph (TEM) image of silica-coated Tbₓ[c,c,t-Pt(NH₃)₂(Cl)₂(O₂CCH₂CH₂CO₂)₂]_{y} nanoparticles (i.e., NCP-1′) isolated after 3 hours of exposure to a silica-coating solution. The scale bar in the left-hand corner of the image represents 50 nm.

FIG. 16C is a transmission electron micrograph (TEM) image of silica-coated Tbₓ[c,c,t-Pt(NH₃)₂(Cl)₂(O₂CCH₂CH₂CO₂)₂]_{y} nanoparticles (i.e., NCP-1′) isolated after 4 hours of exposure to a silica-coating solution. The scale bar in the left-hand corner of the image represents 50 nm.

FIG. 16D is a transmission electron micrograph (TEM) image of silica-coated Tbₓ[c,c,t-Pt(NH₃)₂(Cl)₂(O₂CCH₂CH₂CO₂)₂]_{y} nanoparticles (i.e., NCP-1′) isolated after 7 hours of exposure to a silica-coating solution. The scale bar in the left-hand corner of the image represents 50 nm.

FIG. 17 is a graph showing the dynamic light scattering (DSS) curves for Tbₓ[c,c,t-Pt(NH₃)₂(Cl)₂(O₂CCH₂CH₂CO₂)₂]_{y} nanoparticles (NCP-1, lighter shaded circles), polyvinylpyrrolidone(PVP)-coated NCP-1 (NCP-1/PVP, more darkly shaded circles), silica-coated-NCP-1 having a 2 nm thick silica coating (NCP-1′-a, triangles), and silica-coated-NCP-1 having a 7 nm thick silica coating (NCP-1′-b, darkly shaded squares).

FIG. 18 is a graph showing the thermogravimetric analysis (TGA) curves for the c,c,t-Pt(NH₃)₂(Cl)₂(O₂CCH₂CH₂CO₂)₂ molecular complex (DSCP), Tbₓ[c,c,t-Pt(NH₃)₂(Cl)₂(O₂CCH₂CH₂CO₂)₂]_{y} nanoparticles (NCP-1), and silica-coated Tbₓ[c,c,t-Pt(NH₃)₂(Cl)₂(O₂CCH₂CH₂CO₂)₂]_{y} nanoparticles (NCP-1′).

FIG. 19 is a graph showing the release profiles for Tbₓ[c,c,t-Pt(NH₃)₂(Cl)₂(O₂CCH₂CH₂CO₂)₂]_{y} nanoparticles (NCP-1, circles), silica coated-NCP-1 having a 2 nm thick silica coating (NCP-1′-a, squares) and silica coated-NCP-1 having a 7 nm thick silica coating (NCP-1′-b, triangles) plotted as the % Pt released against time.

FIG. 20 is a graph showing the in vitro cytotoxicity assay curves for human colon cancer cells (HT-29 cells) obtained by plotting the % cell viability against the Pt concentration for cisplatin (darker diamonds), the c,c,t-Pt(NH₃)₂(Cl)₂(O₂CCH₂CH₂CO₂)₂ molecular complex (DSCP, squares), Tbₓ[c,c,t-Pt(NH₃)₂(Cl)₂(O₂CCH₂CH₂CO₂)₂]_{y} nanoparticles (NCP-1, triangles), RGD-targeted, silica-coated (2 nm thickness) Tbₓ[c,c,t-Pt(NH₃)₂(Cl)₂(O₂CCH₂CH₂CO₂)₂]_{y} nanoparticles (NCP-1′-a-c(RGDfK, circles), and RGD-targeted, silica-coated (7 nm thickness) Tbₓ[c,c,t-Pt(NH₃)₂(Cl)₂(O₂CCH₂CH₂CO₂)₂]_{y} nanoparticles (NCP-1′-b-c(RGDfK, lighter shaded diamonds).

FIG. 21A is a graph showing the in vitro cytotoxicity assay curves for human breast cancer cells (MCF-7 cells) obtained by plotting the % cell viability against the Pt concentration for cisplatin (circles) and for the c,c,t-Pt(NH₃)₂(Cl)₂(O₂CCH₂CH₂CO₂)₂ molecular complex (DSCP, diamonds).

FIG. 21B is a graph showing the in vitro cytotoxicity assay curves for human breast cancer cells (MCF-7 cells) obtained by plotting the % cell viability against the Pt concentration for cisplatin (circles) and silica-coated (7 nm thickness) Tbₓ[c,c,t-Pt(NH₃)₂(Cl)₂(O₂CCH₂CH₂CO₂)₂]_{y} nanoparticles (NCP-1′-b, diamonds).

### DETAILED DESCRIPTION

The presently disclosed subject matter will now be described more fully hereinafter with reference to the accompanying examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a metal ion” includes a plurality of such metal ions, and so forth.

Unless otherwise indicated, all numbers expressing quantities of size, number of metal ions, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of size (i.e., diameter), weight, concentration, or percentage is meant to encompass variations of, in some embodiments ±20%, in some embodiments ±10%, in some embodiments ±5%, in some embodiments ±1%, and in some embodiments ±0.1% from the specified amount, as such variations are appropriate to perform the disclosed methods.

The terms “nanoscale,” “nanomaterial,” “nanometer-scale polymer” “nanocluster” and “nanoparticle” refer to a structure having at least one region with a dimension (e.g., length, width, diameter, etc.) of less than about 1,000 nm. In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is less than about 10 nm.

In some embodiments, the nanoparticle is approximately spherical. When the nanoparticle is approximately spherical, the characteristic dimension can correspond to the diameter of the sphere. In addition to spherical shapes, the nanoparticle or other nanoscale material can be disc-shaped, oblong, polyhedral, rod-shaped, cubic, or irregularly-shaped. A nanoscale material can also be irregularly shaped or comprise clusters of spheres, rods, discs, or cubes.

The nanoparticle can comprise an interior region (i.e., the space between the outer dimensions of the particle) and an outer surface (i.e., the surface that defines the outer dimensions of the particle). In some embodiments, the particle can comprise one or more layers. Thus, for example, a spherical nanoparticle can comprise one or more concentric layers, each successive layer being dispersed over the outer surface of the smaller layer closer to the center of the particle. The particle can be solid or porous or can contain a hollow interior region. In some embodiments, the nanoparticle can comprise two layers, an inner core and an outer layer or shell dispersed over the core.

The terms “polymer” and “polymeric” refer to chemical structures that have repeating units (i.e., multiple copies of a given chemical substructure). As used herein, polymers can refer to groups having more than 10 repeating units and/or to groups wherein the repeating unit is other than methylene. Polymers can be formed from polymerizable monomers. A polymerizable monomer is a molecule that comprises one or more reactive moieties {e.g., siloxy ethers, hydroxyls, amines, vinylic groups (i.e., carbon-carbon double bonds), halides (i.e., Cl, Br, F, and I), esters, activated esters, and the like} that can react to form bonds with other molecules. Generally, each polymerizable monomer molecule can bond to two or more other molecules. In some cases, a polymerizable monomer will bond to only one other molecule, forming a terminus of the polymeric material. Exemplary polymerizable monomers include, but are not limited to, acrylamide, acrylic acid, and silyl ethers.

Polymers can be organic, or inorganic, or a combination thereof. As used herein, the term “inorganic” refers to a compound or composition that contains at least some atoms other than carbon, hydrogen, nitrogen, oxygen, sulfur, phosphorous, or one of the halides. Thus, for example, an inorganic polymer can contain one or more silicon atoms or one or more metal atoms in repeating units.

Organic polymers do not include silica or metal atoms in their repeating units. Exemplary organic polymers include polyvinylpyrrolidone (PVO), polyesters, polyamides, polyethers, polydienes, and the like. Some organic polymers contain biodegradable linkages, such as esters or amides, such that they can degrade overtime under biological conditions.

Polymers can also comprise coordination complexes. When the coordination complexes are repeating units in the polymer, the polymer can be referred to as a coordination polymer.

The terms “coordination complex” and “metal coordination complex” refer to a chemical species in which there is a coordinate bond between a metal ion and one or more ligands, wherein each ligand comprises an electron pair donor (i.e., chelating group). Thus, chelating groups are generally electron pair donors, molecules or molecular ions having unshared electron pairs available for donation to a metal ion.

The terms “bonding” or “bonded” and variations thereof can refer to either covalent or non-covalent bonding. In some cases, the term “bonding” refers to bonding via a coordinate bond.

The terms “coordinate bond” or “coordination bond” refer to an interaction between an electron pair donor and a coordination site on a metal ion resulting in an attractive force between the electron pair donor and the metal ion. The use of this term is not intended to be limiting, in so much as certain coordinate bonds also can be classified as have more or less covalent character (if not entirely covalent character) depending on the characteristics of the metal ion and the electron pair donor.

The terms “chelating agent,” “ligand,” “metal coordination ligand,” “chelating group,” and “chelator” refer to a molecule or molecular ion or species having an unshared electron pair available for donation to a metal ion. In some embodiments, the metal ion is coordinated by two or more electron pairs to the chelating agent. The terms “bidentate,” “tridentate,” “tetradentate,” and “pentadentate” refer to chelating agents having two, three, four, and five electron pairs, respectively, available for simultaneous donation to a metal ion coordinated by the chelating agent. In some embodiments, the electron pairs of a chelating agent form coordinate bonds with a single metal ion. In some embodiments, the electron pairs of a chelating agent form coordinate bonds with more than one metal ion, with a variety of binding modes being possible.

The term “nonbridging ligand” refers to a ligand in a coordination polymer that is bonded to a single metal atom. By way of example and not limitation the nonbridging ligand can be monodentate or bidentate. Exemplary nonbridging ligands include, but are not limited to, NH₃, primary amines, secondary amines, diamines, aromatic amines, halides, hydroxide, thiols, and water.

The term “bridging ligand” refers to a ligand in a coordination polymer that is bonded to more than one metal atom (including, but not limited to, metal atoms in more than one repeating metal complex of the polymer). In some embodiments, a bridging ligand can refer to a ligand that is bonded to the platinum metal atoms of two different platinum metal complexes in a coordination polymer comprising a plurality of platinum metal complexes. In some embodiments, the bridging ligand is bonded to the platinum metal atom of one platinum metal complex in a coordination polymer comprising a plurality of platinum metal complexes and at least one other additional metal atom.

The term “ligand” can also refer to biological ligands, such as chemical entities (i.e., groups or whole molecules) that bind to biologically relevant receptors.

The term “cancer” as used herein refers to diseases caused by uncontrolled cell division and the ability of cells to metastasize, or to establish new growth in additional sites. The terms “malignant”, “malignancy”, “neoplasm”, “tumor,” “cancer” and variations thereof refer to cancerous cells or groups of cancerous cells.

Specific types of cancer include, but are not limited to, skin cancers (e.g., melanoma), connective tissue cancers (e.g., sarcomas), adipose cancers, breast cancers, head and neck cancers, lung cancers (e.g., mesothelioma), stomach cancers, pancreatic cancers, ovarian cancers, cervical cancers, uterine cancers, anogenital cancers (e.g., testicular cancer), kidney cancers, bladder cancers, colon cancers, prostate cancers, central nervous system (CNS) cancers, retinal cancer, blood, neuroblastomas, multiple myeloma, and lymphoid cancers (e.g., Hodgkin's and non-Hodgkin's lymophomas).

The terms “anticancer drug” and “anticancer prodrug” refer to drugs or prodrugs known to, or suspected of being able to treat a cancer (i.e., to kill cancer cells, prohibit proliferation of cancer cells, or treat a symptom related to cancer).

The term “imaging agent” refers to a chemical moiety that aids in the visualization of a sample. For example, an imaging agent can be a “contrast agent”, and can refer to a moiety (a specific part of or an entire molecule, macromolecule, coordination complex, or nanoparticle) that increases the contrast of a biological tissue or structure being examined. The contrast agent can increase the contrast of a structure being examined using magnetic resonance imaging (MRI), optical imaging, positron emission tomography (PET) imaging, single photon emission computed tomography (SPECT) imaging, or a combination thereof (i.e., the contrast agent can be multimodal).

The terms “MRI contrast agent” or “MRI imaging agent” refer to a moiety that effects a change in induced relaxation rates of water protons in a sample. MRI contrast agents typically employ paramagnetic metal ions to effect such changes.

As used herein, the term “paramagnetic metal ion” refers to a metal ion that is magnetized parallel or antiparallel to a magnetic field to an extent proportional to the field. Generally, paramagnetic metal ions are metal ions that have unpaired electrons. Paramagnetic metal ions can be selected from the group (including, but not limited to, transition and inner transition elements, including, but not limited to, scandium, titanium, vanadium, chromium, cobalt, nickel, copper, molybdenum, ruthenium, cerium, praseodymium, neodymium, promethium, samarium, europium, terbium, holmium, erbium, thulium, and ytterbium. In some embodiments, the paramagnetic metal ions can be selected from the group (including, but not limited to, gadolinium III (i.e., Gd⁺³ or Gd(III)); manganese II (i.e., Mn⁺² or Mn(II)); copper II (i.e., Cu⁺² or Cu(II)); chromium III (i.e., Cr⁺³ or Cr(III)); iron II (i.e., Fe⁺² or Fe(II)); iron III (i.e., Fe⁺³ or Fe(III)); cobalt II (i.e., Co⁺² or Co(II)); erbium II (i.e., Er⁺² or Er(II)), nickel II (i.e., Ni⁺² or Ni(II)); europium III (i.e., Eu⁺³ or Eu(III)); yttrium III (i.e., Yt⁺³ or Yt(III)); and dysprosium III (i.e., Dy⁺³ or Dy(III)). In some embodiments, the paramagnetic ion is the lanthanide atom Gd(III), due to its high magnetic moment, symmetric electronic ground state, and its current approval for diagnostic use in humans.

The terms “optical imaging agent” or “optical contrast agent” refer to a group that can be detected based upon an ability to absorb, reflect or emit light (e.g., ultraviolet, visible, or infrared light). Optical imaging agents can be detected based on a change in amount of absorbance, reflectance, or fluorescence, or a change in the number of absorbance peaks or their wavelength maxima. Thus, optical imaging agents include those which can be detected based on fluorescence or luminescence, including organic and inorganic dyes.

“Luminescence” occurs when a molecule (or other chemical species) in an electronically excited state relaxes to a lower energy state by the emission of a photon. The luminescent agent in one embodiment can be a chemiluminescent agent. In chemiluminescence, the excited state is generated as a result of a chemical reaction, such as lumisol and isoluminol. In photoluminescence, such as fluorescence and phosphorescence, an electronically excited state is generated by the illumination of a molecule with an external light source. Bioluminescence can occur as the result of action by an enzyme, such as luciferase. In electrochemiluminescence (ECL), the electronically excited state is generated upon exposure of the molecule (or a precursor molecule) to electrochemical energy in an appropriate surrounding chemical environment. Examples of electrochemiluminescent agents are provided, for example, in U.S. Pat. Nos. 5,147,806 and 5,641,623; and in U.S. Patent Application Publication No. 2001/0018187; and include, but are not limited to, metal cation-liquid complexes, substituted or unsubstituted polyaromatic molecules, and mixed systems such as aryl derivatives of isobenzofurans and indoles. The electrochemiluminescent chemical moiety can comprise, in a specific embodiment, a metal-containing organic compound wherein the metal is selected from the group consisting of ruthenium, osmium, rhenium, iridium, rhodium, platinum, palladium, molybdenum, technetium and tungsten.

As described above, the term “fluorophore” refers to a species that can be excited by visible light or non-visible light (e.g., UV light). Examples of fluorophores include, but are not limited to: quantum dots and doped quantum dots (e.g., a semiconducting CdSe quantum dot or a Mn-doped CdSe quantum dot), fluorescein, fluorescein derivatives and analogues, indocyanine green, rhodamine, triphenylmethines, polymethines, cyanines, phalocyanines, naphthocyanines, merocyanines, lanthanide complexes or cryptates, fullerenes, oxatellurazoles, LaJolla blue, porphyrins and porphyrin analogues and natural chromophores/fluorophores such as chlorophyll, carotenoids, flavonoids, bilins, phytochrome, phycobilins, phycoerythrin, phycocyanines, retinoic acid and analogues such as retinoins and retinates.

The term “quantum dot” refers to semiconductors comprising an inorganic crystalline material that is luminescent (i.e., that is capable of emitting electromagnetic radiation upon excitation). The quantum dot can include an inner core of one or more first semiconductor materials that is optionally contained within an overcoating or “shell” of a second semiconductor material. A semiconductor nanocrystal core surrounded by a semiconductor shell is referred to as a “core/shell” semiconductor nanocrystal. The surrounding shell material can optionally have a bandgap energy that is larger than the bandgap energy of the core material and can be chosen to have an atomic spacing close to that of the core substrate.

Suitable semiconductor materials for quantum dots include, but are not limited to, materials comprising a first element selected from Groups 2 and 12 of the Periodic Table of the Elements and a second element selected from Group 16. Such materials include, but are not limited to ZnS, ZnSe, ZnTe, CDs, CdSe, CdTe, HgS, HgSe, HgTe, MgS, MgSe, MgTe, CaS, CaSe, CaTe, SrS, SrSe, SrTe, BaS, BaSe, BaTe, and the like. Suitable semiconductor materials also include materials comprising a first element selected from Group 13 of the Periodic Table of the Elements and a second element selected from Group 15. Such materials include, but are not limited to, GaN, GaP, GaAs, GaSb, InN, InP, InAs, InSb, and the like. Semiconductor materials further include materials comprising a Group 14 element (Ge, Si, and the like); materials such as PbS, PbSe and the like; and alloys and mixtures thereof. As used herein, all reference to the Periodic Table of the Elements and groups thereof is to the new IUPAC system for numbering element groups, as set forth in the Handbook of Chemistry and Physics, 81st Edition (CRC Press, 2000).

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl)hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

As used herein, the term “acyl” refers to an organic carboxylic acid group wherein the —OH of the carboxyl group has been replaced with another substituent. Thus, an acyl group can be represented by RC(═O)—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

“Alkoxyl” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangably with “alkoxyl”.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Ethers” are groups of the structure —R—O—R—, wherein each R is alkyl, substituted alkyl, aryl, aralkyl, or substituted aryl.

“Carbamoyl” refers to an H₂N—C(═O)— group. An “amide” is a compound of the structure H₂N—C(═O)—R or RHN—C(═O)—R, or R₂N—C(═O)—R, wherein each R is independently alkyl, substituted alkyl, aryl, aralkyl, or substituted aryl. A “carbamate” is a compound of the structure H₂N—C(═O)—OR or RHN—C(═O)—OR, or R₂N—C(═O)—OR, wherein each R is independently alkyl, substituted alkyl, aryl, aralkyl, or substituted aryl.

The term “amine” refers to NH₃, primary amines (i.e., compounds of the structure H₂NR, wherein R is alkyl, substituted alkyl, aryl, aralkyl, or substituted aryl), secondary amines (i.e., compounds of the structure HNR₂, wherein each R is independently alkyl, substituted alkyl, aryl, aralkyl, or substituted aryl or wherein the two R groups together are an alkylene group), and tertiary amines (i.e., compounds of the structure NR₃, wherein each R is independently alkyl, substituted alkyl, aryl, aralkyl, or substituted aryl or wherein two R groups together are an alkylene group). The term amine also refers to aromatic amines, including aromatic compounds having a nitrogen atom in the aromatic ring, such as, but not limited to, pyridine, bipyridines, indole, pyrimidine, and the like, as well as amines with aromatic substituents (e.g., aniline).

The terms “carboxylic acid” and “carboxylate” refer to the —C(═O)OH or —C(═O)O⁻ group. As will be understood by one of skill in the art, the protonation state of the group will vary according to the chemical environment.

The term “ester” refers to the —C(═O)OR group, wherein R can be alkyl, substituted alkyl, cycloalkyl, aryl, substituted aryl, aralkyl, and the like. A “carbonate” is a compound of the structure RO—C(═O)—OR wherein each R is independently alkyl, substituted alkyl, aryl, aralkyl, or substituted aryl.

The terms “halo”, “halide”, or “halogen” as used herein refer to fluoro, chloro, bromo, and iodo groups.

The terms “hydroxyl” and “hydroxyl” refer to the —OH group.

The terms “hydroxyalkyl” or “hydroxy-substituted alkyl” refer to an alkyl group substituted with an —OH group.

The terms “mercapto” or “thiol” refer to the —SH group. A “thioether” refers to a —R—S—R group wherein each R is independently alkyl or aryl.

The term “oxo” refers to a compound described previously herein wherein a carbon atom is replaced by an oxygen atom.

The term “phosphate” refers to the —P(═O)(OR)₃ group, wherein R can be H, alkyl, aralkyl, aryl, or a negative charge.

The term “phosphonate” refers to the —P(═O)(OR)₂ group, wherein R can be H, alkyl, aralkyl, aryl, or a negative charge.

The term “alkene” refers to a group having a carbon-carbon double bond.

The term “alkyne” refers to a group having a carbon-carbon triple bond.

The term “silyl” refers to groups comprising silicon atoms (Si).

As used herein, the terms “siloxy” and “silyl ether” refer to groups or compounds including a silicon-oxygen (Si—OR) bond. In some embodiments, the terms refer to compounds comprising one, two, three, or four alkoxy, aralkoxy, or aryloxy groups bonded to a silicon atom. Each alkyloxy, aralkoxy, or aryloxy group can be the same or different.

The term “silanol” refers to the Si—OH group.

The term “poly(siloxane)” refers to a polymeric group of the formula R₂SiO, wherein R is H, alkyl, aralkyl, or aryl.

The term “poly(silsesquioxane)” refers to a polymeric group of the formula RSiO_{1.5}, wherein R is H, alkyl, aralkyl, or aryl.

When the term “independently selected” is used, the groups being referred to (e.g., the bridging ligands, nonbridging ligands, linking moieties, etc.), can be identical or different.

The term “diaqua complex” refers to a metal coordination complex comprising two water ligands.

By “nonplatinum” it is meant an atom, chemical moiety, metal complex, or molecule that does not contain a platinum atom.

The term “hydrophilic” refers to the ability of a molecule or chemical species to interact with water. Thus, hydrophilic molecules are typically polar or have groups that can hydrogen bond to water. The term “hydrophobic” refers to a molecule that interacts poorly with water (e.g., does not dissolve in water or does not dissolve in water to a large extent).

The term “lipophilic” refers to a molecule or chemical species that interacts (e.g., dissolves in) fat or lipids.

The term “amphiphilic” refers to a molecule or species that has both hydrophilic and hydrophobic (or lipophilic) attributes.

### II. Nanoscale Coordination Polymers as Anticancer Therapeutic Agents

Nanoscale coordination polymers (NCPs) are a class of materials constructed from metal complexes having polydentate bridging ligands that can form coordination bonds to metals in more than one metal complex. Due to their inherent solubility in aqueous environments, NCPs can be designed to facilitate the controlled release of therapeutic groups, such as anticancer therapeutic agents. For example, anticancer therapeutic agents or their prodrugs can be bridging ligands in coordination polymers that make up the NCPs or can be the metal complexes, themselves.

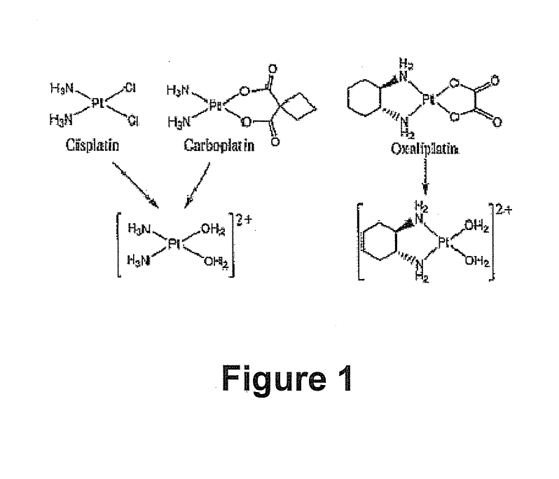

Several currently used anticancer prodrugs involve platinum metal complexes. Some examples of platinum-based anticancer prodrugs, i.e., cisplatin, carboplatin, and oxaliplatin, are shown in FIG. 1. They are used for treating testicular, ovarian, bladder, head and neck, esophageal, small and non-small cell lung, breast, cervical, stomach and prostate cancers, as well as Hodgkin's and non-Hodgkin's lymphomas, neuroblastoma, sarcomas, multiple myeloma, melanoma, and mesothelioma. These platinum-containing anticancer prodrugs convert to the highly potent platinum-aqua complexes inside cells. The prodrugs currently used, including those shown in FIG. 1, are not specific to cancer cells and can taken up by noncancerous cells, leading to many severe side effects including nausea and vomiting, kidney toxicity, blood test abnormalities, low white blood cell count, low red blood cell count, peripheral neuropathy, hearing loss, and hair loss. Often these side effects limit the dose and, thus, the therapeutic efficiency of platinum-based anticancer drugs. As described further hereinbelow, stabilized NCPs can be prepared which are designed for sustained release of anticancer platinum-based prodrugs upon delivery to an intended tissue or tissues.

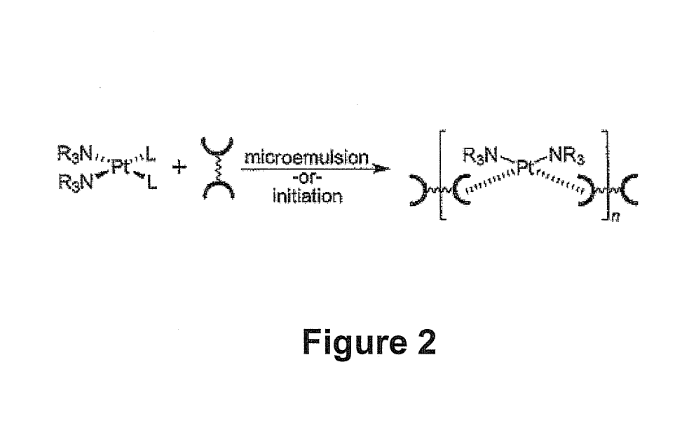

Thus, in some embodiments, the presently disclosed subject matter provides novel coordination polymers (and nanomaterials thereof) comprising coordination polymers that comprise a plurality of platinum metal complexes. For example, as shown in FIG. 2, Pt(II) complexes with good leaving groups, L (e.g., L=water) can be linked by organic bridging ligands to form coordination polymer nanoparticles that carry a high payload of cytotoxic Pt(II) complexes. As described further hereinbelow, the coordination polymer nanoparticles can be prepared by the addition of an initiating (i.e., “poor”) solvent to a precursor solution containing a cis-(NR₃)₂PtL₂ complex, which can be dicationic or neutral depending on the charge carried by the L ligands, and the organic bridging ligands. Alternatively, the nanoparticles can be obtained under reverse microemulsion conditions. In some embodiments, the bridging ligands can be further linked or crosslinked by a second metal center (i.e., M³⁺ or M²⁺) such as lanthanide (Ln³⁺) or a non-platinum transition metal (e.g., Zn²⁺) to form a bimetallic coordination polymer. In some embodiments, the coordination polymers can comprise a plurality of Pt(II) metal complexes wherein the plurality of metal complexes comprise two or more different Pt(II) metal complexes (e.g., two or more different Pt(II) anticancer prodrugs).

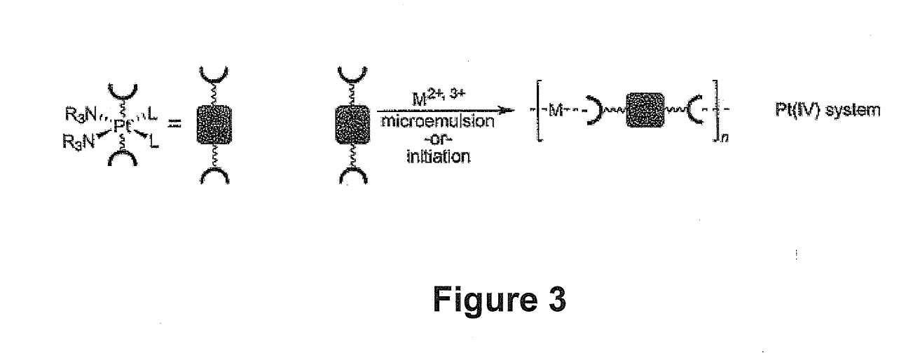

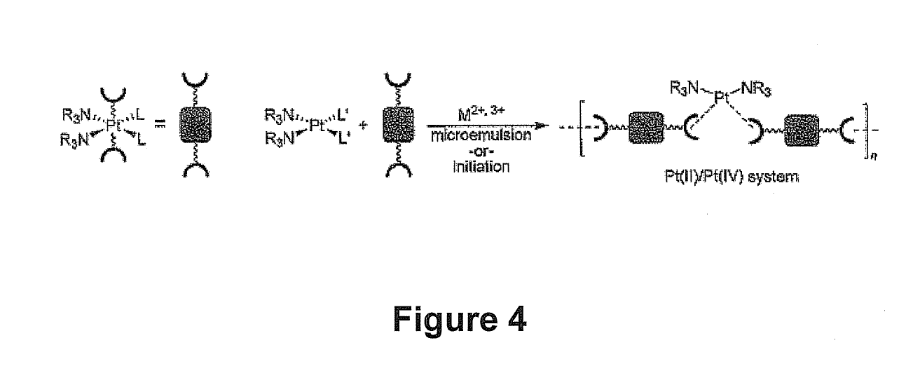

Pt(IV) complexes can also be used as prodrugs for cancer therapy. These Pt(IV) complexes can be readily reduced to Pt(II) species such as cisplatin and oxaliplatin under physiological conditions, and they can be further transformed to their corresponding highly cytotoxic Pt(II) bis(aqua) complexes. Pt(IV) complexes, such as c,c,t-Pt(NH₃)₂Cl₂(O₂CCH₂CH₂CO₂H)₂ (i.e., disuccinatocisplatin (DSCP)) can be incorporated into coordination polymers. As with Pt(II) metal complex-based coordination polymers, Pt(IV) metal complex-based coordination polymers can also be further linked or crosslinked by a second metal center such as a lanthanide metal (Ln³⁺) or a non-platinum transition metal (e.g., Zn²⁺) to form bimetallic coordination polymer nanoparticles. As shown in FIG. 3, coordination polymers comprising a plurality of Pt(IV) complexes can be formed via microemulsion or precipitation with a initiator solvent. The coordination polymers or their NCPs can comprise a plurality of Pt(II) metal complexes wherein the plurality of metal complexes comprise two of more different Pt(II) metal complexes Further, as shown in FIG. 4, coordination polymers and NCPs of the presently disclosed subject matter can comprise a mixture of Pt(II) and Pt(IV) metal complexes.

In some embodiments, the presently disclosed subject matter provides a nanoparticle comprising a coordination polymer comprising a plurality of platinum metal complexes wherein one or more of the platinum metal complexes each comprises:
a platinum metal atom;
at least one nonbridging ligand, wherein the at least one nonbridging ligand is bonded to the platinum metal atom through at least one coordination bond; and
at least one bridging ligand, wherein the at least one bridging ligand is bonded to the platinum metal atom through at least one coordination bond and comprises at least one linking moiety, wherein each of the at least one linking moiety is bonded to an additional metal atom via a coordination bond.

Accordingly, the presently disclosed subject matter provides nanoparticles comprising coordination polymers that act as prodrugs of platinum-based anti-cancer therapeutics. When present in aqueous environments, the coordination polymers “dissolve” (i.e., one or more ligands of individual platinum metal complexes can be replaced by water). If the ligands replaced by water include the bridging ligands, one or more of the individual platinum metal complexes are released from the polymer, either in a biologically active form, or in a form which can be further reacted with water to provide a biologically active (e.g., cytotoxic) form of the complex (e.g., a Pt(II) bis(aqua) species).

The linking moieties of the bridging ligands can include any group that forms a coordination bond with a metal atom. For example, linking moieties can include, but are not limited to carboxylates, carboxylic acids, esters, amides, carbonates, carbamates, amines (e.g., primary amines, secondary amines or tertiary amines), hydroxyls (e.g., hydroxy-substituted alkyls or hydroxy-substituted aryls), thiols, thioethers, phosphates, phosphonates, and ethers. In some embodiments, each linking moiety present in a bridging ligand is independently selected from the group consisting of a carboxylate, a carboxylic acid, an amine, and an amide.

Each of the at least one bridging ligand is bonded via one or more coordination bond to the platinum metal atom of one of the plurality of platinum metal complexes in the coordination polymer and to one additional metal atom, which can be the platinum metal atom of a second platinum metal complex in the coordination polymer. In such cases, the same bridging ligand can be part of at least two of the plurality of platinum metal complexes. Alternatively, the additional metal atom can be a nonplatinum metal atom (e.g., a lanthanide, actinide, or nonplatinum transition metal atom). Thus, the bridging ligands serve to link and/or crosslink the monomer units (i.e., the individual platinum metal complexes) of the coordination polymer to one another, either directly or through a second metal atom.

Bridging ligands can be nonpolymeric or polymeric. In some embodiments, the coordination polymer of the presently disclosed subject matter comprises only nonpolymeric bridging ligands. By “nonpolymeric bridging ligand” it is meant a bridging ligand having a formula:

Cm-X-Lm,

wherein Cm is a moiety involved in a coordination bond with the platinum metal atom, Lm is a linking moiety, and X is a bivalent group covalently bonded to both Cm and Lm, which contains fewer than 10 repeating units (e.g., fewer than 10, 9, 8, 7, 6, 5, 4, 3, or 2 repeating units). In some embodiments, X can contain only methylene repeating units. In some embodiments, X contains no repeating units. Thus, typically, nonpolymeric bridging ligands can be based on organic small molecules (i.e., molecules having a molecular weight (MW) of less than about 1000, 900, 800, 700, 600, or 500 daltons). As described further, hereinbelow, nonpolymeric bridging ligands can also include synthetic organic or natural product-based anticancer drugs.

In some embodiments, the at least one bridging ligand is monodentate with respect to one or more metal atoms to which it is complexed. For example, the bridging ligand can be bonded to the platinum metal atom of a particular platinum metal complex through one coordination bond and comprises at least one linking moiety for bonding to an additional metal atom. Each of the at least one linking moieties can be bonded to a single additional metal atom (e.g., the platinum metal atom of a second platinum metal complex or a nonplatinum metal atom).

In some embodiments, the bridging ligand comprises at least two carboxylate groups, e.g., wherein one carboxylate group is involved in a coordination bond with the platinum metal complex and the second carboxylate group is involved in a coordination bond with the additional metal atom. The bridging ligand can further comprise one or more additional moieties that are capable of bonding to additional metal atoms via coordination bonds, but which do not necessarily need to be involved in such bonds in the coordination polymer as formed. Thus, suitable bridging ligands include not only dicarboxylates, but also tri-, tetra-, and penta-carboxylates as well. Such multi-carboxylate bridging ligands include, but are not limited to, 1,4-benzene dicarboxylate (i.e., terephthalate); 1,3,5-benzene tricarboxylate, succinate, oxalate, malonate, succinate, glutarate, phthalate, isophthalate, citrate, isocitrate, propane-1,2,3-tricarboxylate, ethylene diamine tetraacetate, and the like. In some embodiments, a platinum metal complex can comprise two bridging ligands selected from the group consisting of 1,4-benzene dicarboxylate (i.e., terephthalate); 1,3,5-benzene tricarboxylate, succinate, oxalate, malonate, succinate, glutarate, phthalate, isophthalate, citrate, isocitrate, ethylene diamine tetraacetate, diethylene triamine pentaacetic acid, diethylenetriamine tetraacetic acid, and propane-1,2,3-tricarboxylate. In some embodiments, the bridging ligand is a benzene dicarboxylate (BDC; e.g., terephthalate, phthalate, isophthalate) or a benzene tricarboxylate (BTC; e.g., 1,3,5-benzene tricarboxylate).

In some embodiments, the at least one bridging ligand can have mixed functionality. Thus, the bridging ligand can be of the formula:

Cm-X-Lm,

wherein Cm is a moiety involved in a coordination bond with the platinum metal atom, Lm is the linking moiety, and X is a bivalent group covalent bonded to both Cm and Lm, wherein Cm and Lm are different types of chemical groups. For example, one of Cm and Lm can be an amine group and the other of Cm and Lm can be carboxylate, carboxylic acid, amide, hydroxyl, or ester. In some embodiments, both Cm and Lm can be amine or both of Cm and Lm can be of the same functionality where the functionality is other than carboxylate or amine.

In some embodiments, the bridging ligand can be multi-dentate (e.g., bidentate) with respect to one or more metal atoms to which it is coordinated. In some embodiments, a platinum metal complex can comprise a single bridging ligand, wherein the single bridging ligand is bonded to the platinum metal atom through two coordination bonds and comprises two linking moieties (e.g. each of which is bonded to a different additional metal atom). In some embodiments, the bridging ligand is a bipyridine dicarboxylate. In some embodiments, the bipyridine dicarboxylate is selected from the group consisting of 2,2′-bipyridine-5,5′-dicarboxylate and 2,2′-bipyridine-4,4′-dicarboxylate.

Nonbridging ligands are ligands that are bonded via coordination bonds to the platinum metal atom of a single platinum metal complex. Thus, nonbridging ligands are ligands that are not bonded to any additional metal atoms. Suitable nonbridging ligands can be independently selected from the group including, but not limited to, NH₃, primary amines, secondary amines, tertiary amines, diamines, halides (i.e., iodide, chloride, bromide, and fluoride), hydroxide, hydroxy-substituted alkyl groups, hydroxy-substituted aryl groups, esters, carboxylates, carboxylic acids, carbamates, thiols, amides, and combinations thereof. Nonbridging ligands can be selected based on the ligands of platinum-based anticancer therapeutics known in the art, such as, but not limited to carboplatin, cisplatin, nedaplatin, oxaliplatin, and satraplatin. In some embodiments, the nonbridging ligands are selected from the group consisting of Cl, NH₃, cyclohexylamine, acetate, hydroxyacetate, cyclohexanediamine, bipyridine, oxylate, malonate, and cyclobutane-1,1-dicarboxylic acid.

Nonbridging ligands can be monodentate or multidentate (e.g., bidentate). Typically, each platinum metal complex can comprise between 1 and 4 nonbridging ligands (i.e., 1, 2, 3, or 4 nonbridging ligands), such that the platinum metal atom is involved in a total of 4 or 6 coordination bonds with the bridging and nonbridging ligands of the complex.

In some embodiments, each of the plurality of platinum metal complexes is independently selected from the group consisting of:
Pt[(NH₃)₂(Cl)₂(O₂CCH₂CH₂CO₂)₂] (i.e., DSCP);
Pt[(NH₃)₂(Cl)₂{O₂CC₆H₃(CO₂)₂}₂];
dichloro(2,2′-bipyridine-4,4′-dicarboxylato)platinum (II);
dichloro(2,2′-bipyridine-5,5′-dicarboxylato)platinum (II); and
P[(NH₃)₂(Cl)₂(ethylene diamine tetraacetate)₂].

The nanoparticles of the presently disclosed subject matter generally have at least one dimension less than about 1000 nm. In some embodiments, the nanoparticles can have at least one dimension less than about 500 nm, less than about 400 nm, less than about 300 nm, or less than about 200 nm. In some embodiments, the nanoparticles are approximately spherical and each have a diameter of about 500 nm or less. In some embodiments, the nanoparticles can each have a diameter of between about 20 nm and about 250 nm. Thus, each nanoparticle can have a diameter of about 20, 30, 40, 50, 60, 70, 80, 90, 100, 110, 120, 130, 140, 150, 160, 170, 180, 190, 200, 210, 220, 230, 240, or 250 nm. In some embodiments, each nanoparticle has a diameter of between about 40 nm and about 70 nm.

### II.A. Nanoscale Coordination Polymers with Nonplatinum Anticancer Drugs as Bridging Ligands

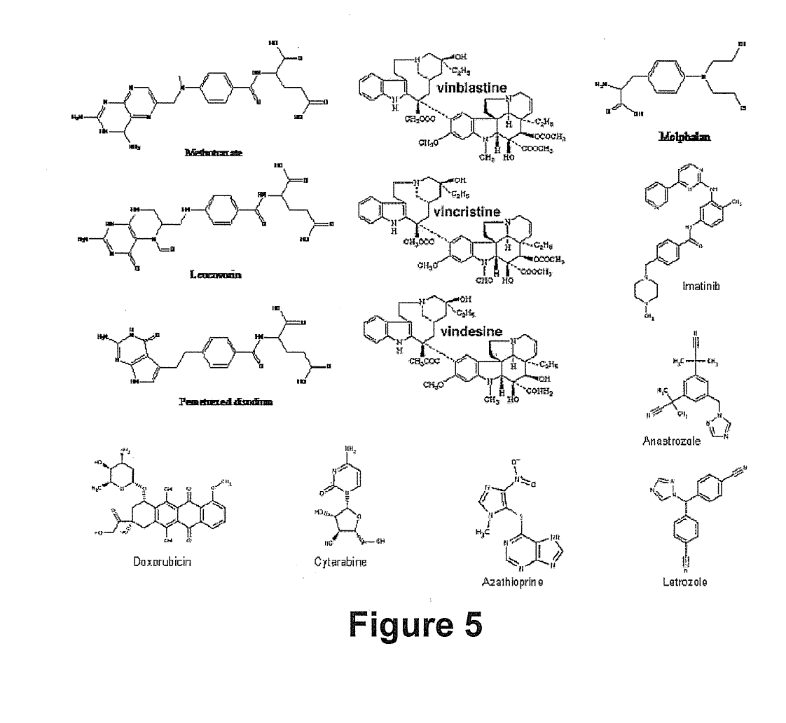

Many currently used anticancer drugs do not contain a metal, or do not contain a platinum metal atom, but do bear functional groups that can coordinate to various metal centers. Some examples of organic (i.e., non-metallic) anticancer drugs are shown in FIG. 5. When appropriate metal centers are used (e.g., either a platinum metal atom or a nonplatinum metal atom), these nonplatinum anticancer drugs or their prodrugs can be linked or crosslinked by the metal atoms to form polymeric species based on metal-ligand coordination bonds. In some embodiments, the presently disclosed subject matter provides coordination polymers comprising nonplatinum (e.g., small molecule, organic) anticancer drugs or prodrugs that are linked or crosslinked via platinum or nonplatinum metal atoms. In some embodiments, the presently disclosed subject matter provides coordination polymers and nanoparticles comprising coordination polymers that comprise both platinum-based anticancer prodrugs and nonplatinum anticancer drugs or prodrugs. The potential synergistic anticancer effects of the platinum-based anticancer drugs and the nonplatinum anticancer drugs can add to the potency of the chemotherapy and can potentially alleviated the acquired drug resistance problem often faced by conventional anticancer drugs.

In some embodiments, a nonplatinum (e.g., small molecule, organic) anticancer drug or prodrug can be used as a bridging ligand in a NCP comprising one or more platinum metal complex. Any nonplatinum anticancer drug or prodrug known in the art and having suitable functionality for coordinating to metal atoms can be used. Suitable nonplatinum anticancer drug bridging ligands include, but are not limited to, methotrexate, folic acid, leucovorin, vinblastine, vincristine, melphalan, imatinib, pemetrexed, vindesine, anastrozole, doxorubicin, cytarabine, azathioprine, letrozole and carboxylates thereof. Thus, when the coordination polymer dissolves in an aqueous environment to provide individual platinum metal complexes, the bridging ligand organic anticancer drugs are also released.

Alternatively or in addition to being incorporated into the coordination polymers as bridging ligands, one or more nonplatinum anticancer drugs or prodrugs can be grafted onto the surface of a NCPs (e.g., a NCPs comprising a plurality of platinum metal complexes). The nonplatinum anticancer drugs can be grafted onto the surface of the NCPs via covalent linkages that are cleavable under biological conditions (e.g., at a certain pH or in response to a reductant present in vivo and/or in vitro). Such linkages can be enzymatically cleavable. If desired, the nonplatinum anticancer therapeutic can be derivatized (e.g., covalently bonded) with a polymeric or oligomeric linker group (e.g., a siloxane or poly(ethylene glycol)) that can be used as a chemical tether between the drug and the nanoparticle.

In some embodiments, the presently disclosed subject matter provides a nanoscale coordination polymer that comprises anticancer drugs or prodrugs and is free of platinum. Thus, in some embodiments, the presently disclosed subject matter provides a coordination polymer comprising a plurality of nonplatinum metal complexes wherein the nonplatinum metal complexes are linked via bridging ligands that comprise nonplatinum anticancer drugs. The nonplatinum anticancer bridging ligands can comprise known organic anticancer drugs, such as, but not limited to those, shown in FIG. 5, or any other known anticancer drug that comprises chemical groups capable of forming coordination bonds with metal atoms. The nonplatinum metal complexes can comprise any suitable metal atom, e.g., a nonplatinum transition metal atom, a lanthanide metal atom, or an actinide metal atom.

### II.B. Nanoscale Coordination Polymers with Polymeric Bridging Ligands

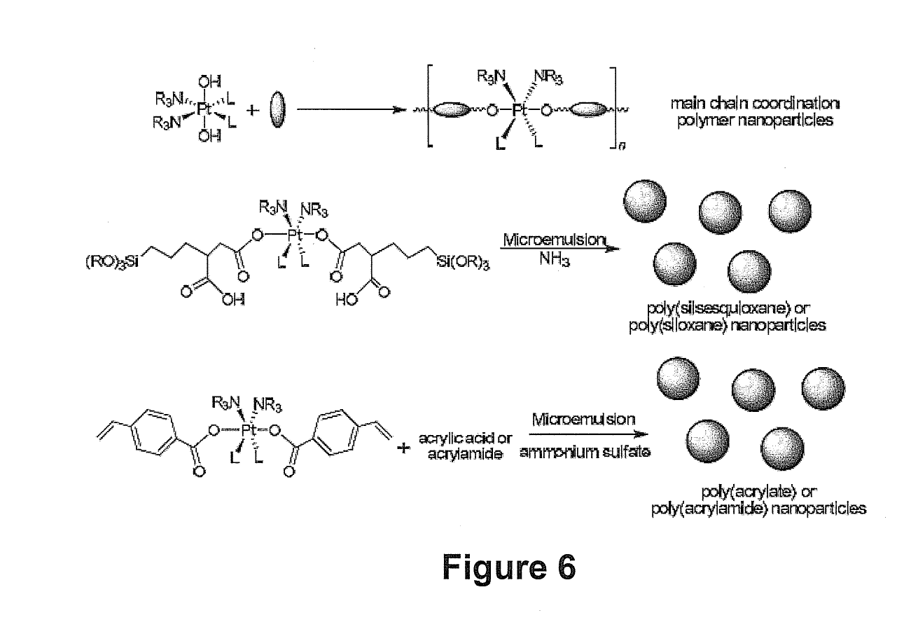

As shown in FIG. 6, in some embodiments, Pt(II) and/or Pt(IV) prodrugs can be polymerized with polymers or polymerizable monomers (i.e., represented by the oval shape in the uppermost reaction shown in FIG. 6) to form copolymers of the coordination metal complexes and other polymers. In some embodiments, the Pt(II) or Pt(IV) prodrugs used in the preparation of NCPs having polymeric bridging ligands comprise a prelinking moiety that has a chemical functional group that can react with a polymerizable monomer. See FIG. 6, middle reaction scheme. Such prelinking moieties include siloxyl functional groups that can react with silyl ethers, poly(siloxanes) or poly(silsesquioxanes). In some embodiments, the Pt(II) or Pt(IV) prodrug contains a functional group (e.g., an alkene) that can react with acrylic acid or acrylamide to form poly(acrylate), poly(acrylamide) or other organic polymer-based linking moieties. See FIG. 6, lower reaction scheme.

Thus, in some embodiments, one or more bridging ligand in an NCP can be polymeric. By polymeric bridging ligand is meant a bridging ligand of the formula:

Cm-[X]ₙ-Lm,

wherein Cm is a moiety involved in a coordination bond with the platinum metal atom, Lm is a linking moiety, n is an integer greater than 1, and X is a nonmethylene bivalent moiety. In some embodiments, X is a non-metallic moiety other than methylene. In some embodiments, X comprises a non-platinum metal atom. In some embodiments, n is greater than 10, 20, 30, 40, 50, 60, 70, 80, 90, or 100. Typically, polymeric bridging ligands can have MWs greater than about 1000. In some embodiments, the polymeric briding ligand comprises poly(siloxane), poly(silsesquioxane), poly(acrylate) or poly(acrylamide).

### II.C. Additional Nanoparticle Components

In some embodiments, the presently disclosed NCPs can include one or more components in addition to the platinum metal complexes, the small molecule nonplatinum cancer drugs or prodrugs, or the combination of platinum metal complexes and small molecule nonplatinum anticancer drugs or prodrugs. Such additional components include photosensitizers, radiosensitizers, radionuclides, imaging agents, passivating agents, stabilizing agents, and targeting agents. These additional components can be incorporated into the nanoparticles as ligands of metal complexes, incorporated into pores in the nanoparticles (e.g., physically trapped in pores via co-precipitation during formation of the nanoparticles, wherein the components are not covalently bound to any metal complex), covalently linked to ligands of metal complexes, or linked via coordination bonds to the platinum or other metals present in the NCPs. In some embodiments, the additional components are grafted (i.e., chemically linked via covalent or coordination bonds) to an outer surface of the NCPs. As necessary, the additional components can include small molecule-based or polymer-based linkers in order to facility grafting or other incorporation of the additional compentent into the NCPs.

In some embodiments, metal-containing imaging agents or radiotherapeutic agents can be used to provide the metal atom of a nanoscale coordination polymer. Thus, an imaging agent or radiotherapeutic agent can be copolymerized with platinum-based anticancer prodrugs and/or small molecule, nonplatinum anticancer drugs or prodrugs.

### III. Nanoscale Coordination Polymers for Combination Chemotherapeutic and Photodynamic Therapy

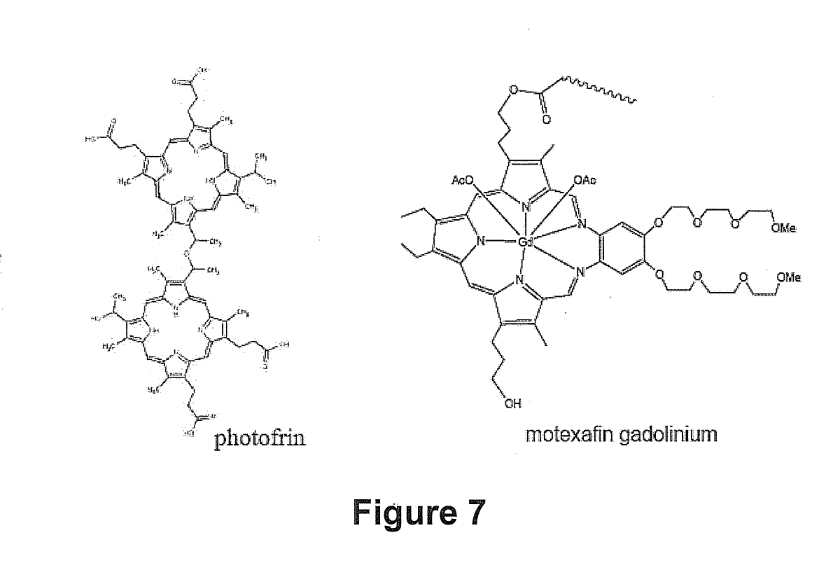

Some anticancer drugs require external stimuli such as intense laser light or X-ray radiation to render them cytotoxic. For example, photosensitizers can be used in combination with light at specific wavelengths to transform triplet state oxygen present in tissues into singlet state oxygen, which can react with nearby biomolecules. PHOTOFRIN® (i.e., porfimer sodium; Axcan Pharma PDT, Inc., Birmingham, Ala., United States of America) has been widely used as a sensitizer for photodynamic therapy (PDT). The chemical structure of PHOTOFRIN® is shown in FIG. 7. The efficacy of the PDT approach to cancer therapy is limited by the lack of efficient methods for accumulating adequate doses of the photosensitizer in the tumor. Other photosensitizers include verteprofin, the chlorins, and 5-aminolevulinic acid.

Thus, in some embodiments, the NCPs can be prepared to include a photosensitizer and be used in conjunction with PDT. In some embodiments, the NCP can be prepared to include the photosensitizer copolymerized with platinum metal complexes. Alternatively, the photosensitizer can be grafted to the surface of an NCP.

### IV. Nanoscale Coordination Polymers for Combination Chemotherapeutic and Radiation Therapy

Radiosensitizers are compounds that can make tumor cells more sensitive to radiation therapy. Metoxafin gadolinium (XCYTRIN®; Pharmacyclics, Sunnyvale, Calif., United States of America) can be used as a prototype radiosensitizer for forming coordination polymer nanoparticles. The structure of metoxafin gadolinium is also shown in FIG. 7. Motexafin gadolinium belongs to the texafrin family of expanded porphyrins. Because of its strong resemblance to porphyrins and other naturally occurring tetrapyrrolic prosthetic groups, the texaphyrins exhibit useful characteristics, such as favorable biolocalization on cancer cells. Two distinct differences of the texaphyrin family from porphyrins make them potentially useful as radiosensitizers. First, the expanded and trianionic nature of the texaphyrin endows a much tighter coordination with large and trivalent lanthanide metals. Second, lanthanide complexes of texaphyrins exhibit unique redox characteristics and act as powerful oxidizing agents (with a reduction potential E_{1/2} of −0.041 for motexafin gadolinium). This latter property can make motexafin gadolinium an efficient X-ray radiation (XRT) enhancing agent (i.e., a radiosensitizer). While dissociated Gd³⁺ has potential toxicity concerns, targeted delivery of motexafin gadolinium in a NCP of the presently disclosed subject matter can enhance the biolocalization of the agent in tumor cells, reducing the dose of motexfin gadolinium needed to achieve optimal therapeutic effect.

Thus, in some embodiments, the NCPs can be prepared to include a radiosensitizer and be used in conjunction with radiation therapy. In some embodiments, the NCP can be prepared to include a radiation source itself. For example, the presently disclosed NCPs can be prepared to include radionuclides (i.e., radioisotopes) that can release radiation upon delivery of the NCP to a targeted cell, tissue or organ. In some embodiments, a beta-emitting metal center (e.g., ⁹⁹Y) can be incorporated as a metal in a NCP either containing Pt-metal complexes or organic anticancer drugs, or both for use in combination chemotherapy and radiotherapy.

### V. Nanoscale Coordination Polymers for Combination Chemotherapy and Imaging

Nanoscale coordination polymer architecture allows for a large variety of contrast agents for different imaging modalities, including, but not limited to magnetic resonance imaging (MRI), optical imaging, positron emission tomography (PET) and single photon emission computed tomography (SPECT). For example, in some embodiments, a paramagnetic metal atom, such as the lanthanide atom Gd³⁺ can be incorporated into the particles (e.g., as an additional metal atom bound via coordination bonds to bridging ligands in bimetallic NCPs) so that the NCPs can be used as dual chemotherapeutic/MRI imaging agents.

Gd³⁺ is often chosen as a metal atom for MRI contrast agents because it has a very high magnetic moment and a symmetric electronic ground state. Transition metals, including, but not limited to, high spin Mn(II) and Fe(III) can also be incorporated into the NCPs to provide MRI contrast enhancement. These metals, when delivered along with anticancer drugs or prodrugs in the NCPs to the tumor sites can allow for high-resolution delineation of the tumors. MR images taken pre- and post-administration of the NCPs can enable assessment of the effectiveness of the anticancer drugs or prodrugs.

In some embodiments, an organic fluorophore (e.g., a near-infrared dye) or luminophore that has a chemical functional group or groups that coordinate to metal atoms can be used in the synthesis of the presently disclosed NCPs. The resulting NCPs can be used as efficient optical imaging contrast agents. Further, radioactive metal centers can be doped into the NCPs for application for PET (e.g., when ⁶⁴Cu is the radioactive metal) or SPECT (e.g., when ¹¹¹In or ^{99m}Tc is the radioactive metal). For example, ⁶⁴Cu can be added to a precursor solution containing non-radioactive transition metals and either Pt-containing or organic anticancer drugs to form an NCP. Given the high sensitivity of the PET imaging technique, only trace amounts of ⁶⁴Cu are needed to allow real-time monitoring of the biodistribution of the NCPs in vivo after administration. In another embodiment, ¹¹¹In and ^{99m}Tc are doped into the NCPs containing Pt-containing or organic anticancer drugs. SPECT can then be used for real-time monitoring of the biodistribution of the NCPs in vivo.

### VI. Stabilization of Nanoscale Coordination Polymers

Because NCPs are soluble in aqueous environments (e.g., biological environments), in some embodiments, it can be desirable to slow down the dissolution of the NCPs (and related release of their anticancer drugs or prodrugs) by the use of a stabilizing agent. The stabilizing agent can be a chemical group, molecule, or delivery vehicle (e.g., liposome or microemulsion) that shields the NCP from the aqueous environment, for example, by reducing or eliminating access of water molecules to the NCP, for a period of time. In some embodiments, the stabilizing agent can be biodegradable, either over time or upon contact with a chemical or biochemical agent.

In some embodiments, the presently disclosed subject matter relates to nanoparticles comprising a core and an outer layer. The core can comprise a coordination polymer. The outer layer, which can surround the core, can comprise an inorganic or organic, shell. For example, the NCP can comprise a coordination polymer core and an outer shell comprising a metal oxide, a silica-based polymer (e.g., silica (SiO₂), a poly(siloxane), or a poly(silsesquioxane)), an organic polymer (e.g., polyvinylpyrrolidone (PVP), a polyamide or a polyester), a lipid bilayer (e.g., a liposome), or combinations thereof. In addition to stabilizing the coordination polymer core, in some embodiments, the stabilizing layer can be further functionalized to impart biological compatibility (e.g., reduced immunogenicity or reduced biological clearance), multimodality (e.g., use as a dual chemotherapeutic/imaging agent, a chemotherapeutic/MRI imaging/optical imaging agent, a dual chemotherapeutic/radiotherapeutic agent, dual chemotherapeutic/PDT agent, dual chemotherapeutic/radiosensitizing agent) and/or specificity (e.g., targeting to a specific type of cell, organ, or tissue in vivo and/or in vitro). The outer layer and the core can be chemically bonded to one another (e.g., via one or more coordination bond or one or more covalent bonds). However, in some embodiments, the outer layer and the core are not chemically bonded to one another, rather, the outer surface or surfaces of the core are merely surrounded or encapsulated by the outer layer material.

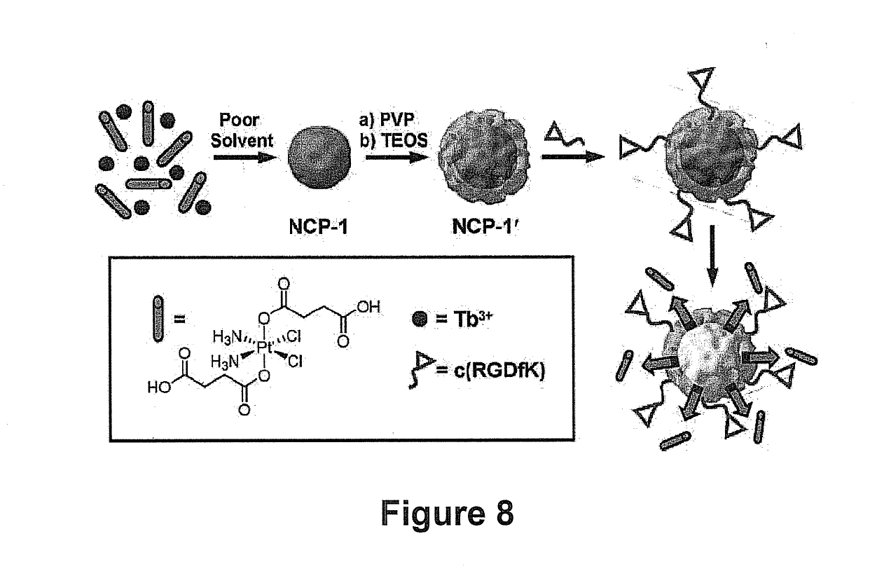

FIG. 8 shows an example of the synthesis of a stabilized NCP. As shown in FIG. 8, an core structure (i.e., NCP-1) comprising a bimetallic coordination polymer is prepared from Pt[(NH₃)₂(Cl)₂(O₂CCH₂CH₂CO₂)₂} and Tb³⁺. The core is then coated with PVP and silica (i.e., from the polymerization of tetraethyl orthosilicate (TEOS)) to provide the silica-modified NCP (i.e., NCP-1′).

### VII. Passivation of Nanoscale Coordination Polymers

The presently disclosed NCPs, particularly those modified with an outer layer, can be functionalized with biocompatible passivating molecules to deter the adsorption of plasma proteins and/or recognition by biological defense systems, such as the reticulo-endothelial system (RES). In some embodiments, the NCP comprises a passivating moiety comprising a polyethylene glycol (PEG)-based polymer. PEG polymers are widely commercially available (e.g., from Aldrich Chemical Company, Milwaukee, Wis., United States of America) in a variety of sizes and with a variety of terminal functionalities to aid in their covalent attachment to the presently disclosed NCPs. PEG is generally hydrophilic, non-biodegradable, and non-immunogenic. The PEG can be any size (e.g., MW of about 500, 1000, 5000, 10,000, 25,000, or more) or polydispersity. In some embodiments, the PEG-based polymer is polyethylene oxide (PEO)-500. In some embodiments, the PEG is grafted onto the outer shell of a stabilized NCP or onto an outer surface of a non-stabilized NCR Other polymers that can be used as passivating agents include, but are not limited to, lipid bilayers (such as liposomes).

### VIII. Targeting of Nanoscale Coordination Polymers

In some embodiments, the NCP can comprise a targeting agent to direct the NCP, once administered, to a target cell, organ, or tissue. In some embodiments, the target is a diseased cell (i.e., a cell associated with at particular disease state, such as a cancer cell). Any targeting moiety known to be located on the surface of the target diseased cells, or expressed by the diseased cells, finds use with the presently disclosed particles. For example, an antibody directed against a cell surface moiety can be used. Alternatively, the targeting moiety can be a ligand directed to a receptor present on the cell surface or vice versa. Thus, targeting moieties include small molecules, peptides, and proteins (including antibodies or antibody fragments (e.g., FABs)).

Targeting moieties for use in targeting cancer cells can be designed around tumor specific antigens including, but not limited to, carcinoembryonic antigen, prostate specific antigen, tyrosinase, ras, HER2, erb, MAGE-1, MAGE-3, BAGE, MN, gp100, gp75, p97, proteinase 3, a mucin, CD81, CID9, CD63; CD53, CD38, CO-029, CA125, GD2, GM2 and O-acetyl GD3, M-TAA, M-fetal or M-urinary find use with the presently disclosed subject matter. Alternatively the targeting moiety can be designed around a tumor suppressor, a cytokine, a chemokine, a tumor specific receptor ligand, a receptor, an inducer of apoptosis, or a differentiating agent. Further, given the importance of the angiogenisis process to the growth of tumors, in some embodiments, the targeting moiety can be developed to target a factor associated with angiogenesis. Thus, the targeting moiety can be designed to interact with known angiogenisis factors such as vascular endothelial growth factor (VEGF). See Brannon-Peppas, L. and Blanchette, J. O., Advanced Drug Delivery Reviews, 56, 1649-1659 (2004).

Tumor suppressor proteins provided for targeting include, but are not limited to, p16, p21, p27, p53, p73, Rb, Wilms tumor (WT-1), DCC, neurofibromatosis type 1 (NF-1), von Hippel-Lindau (VHL) disease tumor suppressor, Maspin, Brush-1, BRCA-1, BRCA-2, the multiple tumor suppressor (MTS), gp95/p97 antigen of human melanoma, renal cell carcinoma-associated G250 antigen, KS 1/4 pan-carcinoma antigen, ovarian carcinoma antigen (CA125), prostate specific antigen, melanoma antigen gp75, CD9, CD63, CD53, CD37, R2, CD81, CO029, TI-1, L6 and SAS. Of course these are merely exemplary tumor suppressors and it is envisioned that the presently disclosed subject matter can be used in conjunction with any other agent that is or becomes known to those of skill in the art as a tumor suppressor.

In some embodiments, targeting is directed to factors expressed by an oncogene. These include, but are not limited to, tyrosine kinases, both membrane-associated and cytoplasmic forms, such as members of the Src family, serine/threonine kinases, such as Mos, growth factor and receptors, such as platelet derived growth factor (PDDG), SMALL GTPases (G proteins) including the ras family, cyclin-dependent protein kinases (cdk), members of the myc family members including c-myc, N-myc, and L-myc and bcl-2 and family members.

Cytokines that can be targeted by the presently disclosed particles include, but are not limited to, IL-1, IL-2, IL-3, IL-4, IL-5, IL-6, IL-7, IL-8, IL-9, IL-10, ILA 1, IL-12, IL-13, IL-14, IL-15, TNF, GM-CSF, β-interferon, and γ-interferon. Chemokines that can be used include, but are not limited to, M1P1α, M1P1β, and RANTES.

Enzymes that can be targeted include, but are not limited to, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, and human thymidine kinase.

Receptors and their related ligands that find use in the context of the presently disclosed subject matter include, but are not limited to, the folate receptor, adrenergic receptor, growth hormone receptor, luteinizing hormone receptor, estrogen receptor, epidermal growth factor (EGF) receptor, fibroblast growth factor receptor (FGFR), and the like. For example, EGF is overexpressed in brain tumor cells and in breast and colon cancer cells. In some embodiments, the targeting moiety is selected from the group consisting of folic acid, guanidine, transferrin, carbohydrates and sugars. In some embodiments, the targeting moiety is a peptide selected from the group consisting of the amino acid sequence RGD and TAT peptides.

In some embodiments, the targeting moiety can comprise cyclic(RGDfK) (i.e., c(RGDfK)). For example, referring again to FIG. 8, a suitably dervitized c(RGDfK) (e.g., a siloxy-functionalized c(RGDfK)) can be grafted onto the outer layer of a silica-modified NCP. In vivo, the targeted NCP can slowly leach out the platinum metal complex after being taken up by a tumor that recognizes the RGD motif. See Scheme 8, final step.

Hormones and their receptors include, but are not limited to, growth hormone, prolactin, placental lactogen, luteinizing hormone, folicle-stimulating hormone, chorionic gonadotropin, thyroid-stimulating hormone, leptin, adrenocorticotropin (ACTH), angiotensin I, angiotensin II, β-endorphin, β-melanocyte stimulating hormone (β-MSH), cholecystokinin, endothelin I, galanin, gastric inhibitory peptide (GIP), glucagon, insulin, amylin, lipotropins, GLP-1 (7-37) neurophysins, and somatostatin.

The presently disclosed subject matter provides that vitamins (both fat soluble and non-fat soluble vitamins) placed in the targeting component of the nanomaterials can be used to target cells that have receptors for, or otherwise take up these vitamins. Particularly preferred for this aspect are the fat soluble vitamins, such as vitamin D and its analogues, Vitamin E, Vitamin A, and the like or water soluble vitamins such as Vitamin C, and the like.

Antibodies can be generated to allow for the targeting of antigens or immunogens (e.g., tumor, tissue or pathogen specific antigens) on various biological targets (e.g., pathogens, tumor cells, and normal tissue). In some embodiments of the presently disclosed subject matter, the targeting moiety is an antibody or an antigen binding fragment of an antibody (e.g., Fab, F(ab′)2, or scFV units). Thus, “antibodies” include, but are not limited to polyclonal antibodies, monoclonal antibodies, chimeric antibodies, single chain antibodies, Fab fragments, and a Fab expression library.

Other characteristics of the nanoparticle also can be used for targeting. Thus, in some embodiments, the enhanced permeability and retention (EPR) effect is used in targeting. The EPR effect is the selective concentration of macromolecules and small particles in the tumor microenvironment, caused by the hyperpermeable vasculature and poor lymphatic drainage of tumors. To enhance EPR, in some embodiments, the exterior of the particle can be coated with or conjugated to a hydrophilic polymer to enhance the circulation half-life of the particle and to discourage the attachment of plasma proteins to the particle.

For additional exemplary strategies for targeted drug delivery, in particular, targeted systems for cancer therapy, see Brannon-Peppas, L. and Blanchette, J. O., Advanced Drug Delivery Reviews, 56, 1649-1659 (2004) and U.S. Pat. No. 6,471,968, each of which is incorporated herein by reference in its entirety.

As necessary, targeting agents can be chemically derivatized to incorporate chemical groups that can be used to graft or otherwise incorporate the targeting agents into NCPs. For example, the targeting agents can be covalently bound to coordination complexes that can be copolymerized into the NCPs. Alternatively, the targeting agents can be derivatized with chemical groups that include functionalities that can react with stabilizing agents or layers surrounding NCPs.

### IX. Formulations

The compositions of the presently disclosed subject matter comprise in some embodiments a composition that includes a NCP and a pharmaceutically acceptable carrier. Any suitable pharmaceutical formulation can be used to prepare the compositions for administration to a subject. In some embodiments, the composition and/or carriers can be pharmaceutically acceptable in humans.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the subject; and aqueous and non-aqueous sterile suspensions that can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are sodium dodecyl sulfate (SDS), in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

In some embodiments, the presently disclosed NCPs can be formulated in liposomes or microemulsions by methods known in the art.

It should be understood that in addition to the ingredients particularly mentioned above, the formulations of this presently disclosed subject matter can include other agents conventional in the art having regard to the type of formulation in question. For example, sterile pyrogen-free aqueous and non-aqueous solutions can be used.

### X. Subjects

The methods and compositions disclosed herein can be used on a sample either in vitro (for example, on isolated cells or tissues) or in vivo in a subject (i.e. living organism, such as a patient). In some embodiments, the subject is a human subject, although it is to be understood that the principles of the presently disclosed subject matter indicate that the presently disclosed subject matter is effective with respect to all vertebrate species, including warm-blooded vertebrates, such as mammals and birds, which are intended to be included in the terms “subject” and “patient”. Moreover, a mammal is understood to include any mammalian species for which employing the compositions and methods disclosed herein is desirable, particularly agricultural and domestic mammalian species.

More particularly provided is anticancer therapeutic or anticancer therapeutic/imaging methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Bird subjects include those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment or imaging of livestock including, but not limited to domesticated swine (pigs and hogs), ruminants, horses, poultry, and the like.

### XI. Administration

Suitable methods for administering to a subject a composition of the presently disclosed subject matter include, but are not limited to, systemic administration, parenteral administration (including intravascular, intramuscular, intraarterial administration), oral delivery, buccal delivery, subcutaneous administration, inhalation, intratracheal installation, surgical implantation, transdermal delivery, local injection, and hyper-velocity injection/bombardment. Where applicable, continuous infusion can enhance drug accumulation at a target site (see, for example, U.S. Pat. No. 6,180,082).

The particular mode of drug administration used in accordance with the methods of the presently disclosed subject matter depends on various factors, including but not limited to the agent and/or carrier employed, the severity of the condition to be treated or treated and imaged, and mechanisms for metabolism or removal of the active agent following administration. For example, relatively superficial tumors can be injected intratumorally. Internal tumors can be treated or treated and imaged following intravenous injection. In some embodiments, selective delivery of a composition to a target is accomplished by intravenous injection of the composition followed by hyperthermia treatment of the target.

For delivery of compositions to pulmonary pathways, compositions of the presently disclosed subject matter can be formulated as an aerosol or coarse spray. Methods for preparation and administration of aerosol or spray formulations can be found, for example, in U.S. Pat. Nos. 5,858,784; 6,013,638; 6,022,737; and 6,136,295.

### XII. Doses

An effective dose of a composition of the presently disclosed subject matter is administered to a subject or to a biological sample (e.g., a sample containing cancer cells). An “effective amount” is an amount of the composition sufficient to produce an anticancer therapeutic effect (e.g., reduction of tumor size, reduction in cancer cell proliferation, cancer cell death, etc.). Actual dosage levels of constituents of the compositions of the presently disclosed subject matter can be varied so as to administer an amount of the composition that is effective to achieve the desired effect for a particular subject and/or target. The selected dosage level can depend upon the activity of the composition and the route of administration.

After review of the disclosure herein of the presently disclosed subject matter, one of ordinary skill in the art can tailor the dosages to an individual subject, taking into account the particular formulation, method of administration to be used with the composition, and nature of the target to be imaged and/or treated. Such adjustments or variations, as well as evaluation of when and how to make such adjustments or variations, are well known to those of ordinary skill in the art.

### XIII. Methods of Use

In some embodiments, the presently disclosed subject matter provides methods of treating cancer comprising the administration of the presently disclosed NCPs (e.g., NCPs comprising coordination polymers comprising a plurality of platinum metal complexes; NCPs comprising coordination polymers comprising anticancer therapeutic bridging ligands, or NCPs comprising coordination polymers comprising platinum metal complexes and anticancer therapeutic bridging ligands). In some embodiments, the NCP can be used in methods involving the treatment of cancer and the imaging of a cell, tissue, or organ. In some embodiments, the NCP can be used in a method of treating cancer that further comprises PDT or radiotherapy. In some embodiments, the NCP comprises a targeting agent and the method comprises delivery of the NCP to a specific cell, tissue or organ (e.g., a cell, tissue or organ associated with a particular disease, such as cancer).

Thus, in some embodiments, the presently disclosed subject matter provides a method of inhibiting proliferation of a cancer cell, the method comprising contacting the cancer cell with a nanoparticle, wherein the nanoparticle comprises a coordination polymer comprising a plurality of platinum metal complexes. By “inhibiting proliferation of a cancer cell” is meant that contacting the cancer cell with the nanoparticle inhibits cell division of the cancer cell. In some embodiments, inhibiting proliferation can further comprise triggering apoptosis in the cancer cell or otherwise causing the death of the cancer cell.

The cancer cell can be any cancer cell. Cancer cells treatable by the presently disclosed methods include, but are not limited to, skin cancer cells, connective tissue cancer cells, breast cancer cells, lung cancer cells, esophogeal cancer cells, stomach cancer cells, a head and neck cancer cell, pancreatic cancer cells, ovarian cancer cells, cervical cancer cells, uterine cancer cells, anogenital cancer cells, kidney cancer cells, bladder cancer cells, colon cancer cells, prostate cancer cells, retinal cancer cells, central nervous system cancer cells, and lymphoid cancer cells. In some embodiments, the cancer cell is selected from the group consisting of a breast cancer cell and a colon cancer cell.

The NCP can comprise a plurality of platinum (II) metal complexes, a plurality of platinum (IV) metal complexes, or a combination thereof. One or more of the platinum metal complexes can comprise:
a platinum metal atom;
at least one nonbridging ligand, wherein the at least one nonbridging ligand is bonded to the platinum metal atom through at least one coordination bond; and
at least one bridging ligand, wherein the at least one bridging ligand is bonded to the platinum metal atom through at least one coordination bond and comprises at least one linking moiety, wherein each of the at least one linking moiety is bonded to an additional metal atom via a coordination bond.

Suitable linking moieties include, but are not limited to, carboxylate, carboxylic acid, ester, carbamate, carbonate, amine (e.g., primary, secondary, tertiary, or aromatic amines), amide, hydroxyl, thiol, ether, thioether, phosphonate, and phosphate. Nonbridging ligands can be independently selected from the group including, but not limited to, NH₃, primary amines, secondary amines, tertiary amines, diamines, aromatic amines, halides, hydroxy-substituted alkyl, hydroxy-substituted aryl, esters, carboxylates, carboxylic acids, carbamates, amides, thiols, hydroxide, and combinations thereof.

In some embodiments, the bridging ligand can comprise at least two carboxylate groups. Bridging ligands can be either polymeric or nonpolymeric. In some embodiments, the NCP comprises only nonpolymeric bridging ligands.

The NCP can comprise one or more platinum metal complex(es) that comprise(s) two bridging ligands, where each of the two bridging ligands is bonded to the platinum metal atom through one coordination bond and comprises at least one linking moiety. Each of the two bridging ligands can optionally be selected from a BDC or BTC. In some embodiments, each of the two bridging ligands can be independently selected from the group including but not limited to 1,4-benzene dicarboxylate; 1,3,5-benzene tricarboxylate; succinate; and ethylene diamine tetraacetate.

The NCP can comprise one or more platinum metal complex(es) that comprise(s) one bridging ligand, where the one bridging ligand is bonded to the platinum metal atom through two coordination bonds and comprises at least two linking moieties. In some embodiments, the bridging ligand is a bipyridine dicarboxylate, such as, but not limited to, 2,2′-bipyridine-5,5′-dicarboxylate and 2,2′-bipyridine-4,4′-dicarboxylate.

In some embodiments, the NCP comprises one or more platinum metal complex(es) that comprise(s) a bridging ligand where the bridging ligand is a nonplatinum anticancer drug or anticancer prodrug. Suitable nonplatinum anticancer drug bridging ligands include, but are not limited to, methotrexate, folic acid, leucovorin, vinblastine, vincristine, melphalan, imatinib, pemetrexed, vindesine, anastrozole, doxorubicin, cytarabine, azathioprine, letrozole and carboxylates thereof.

In some embodiments, the NCP can comprise at least one polymeric bridging ligand. Polymeric bridging ligands can comprise polymers such as, but not limited to, poly(silsesquioxane), poly(siloxane), poly(acrylate) and poly(acrylamide).

In some embodiments, the NCP is bimetallic and includes one or more additional nonplatinum metal atom in addition to the platinum metal atoms of the platinum metal complexes. The nonplatinum metal atoms can include nonplatinum transition metal atoms, lanthanide metal atoms, and actinide metal atoms. For example, the NCP can be a bimetallic coordination polymer comprising formulas such as Tbₓ[Pt(L)ₙ(BL)ₘ]_{y} or Znₓ[Pt(L)ₙ(BL)ₘ]_{y}, wherein each L is a nonbridging ligand and each BL is a bridging ligand.

In some embodiments, each of the plurality of platinum metal complexes in the NCP can be independently selected from the group including but not limited to:
Pt[(NH₃)₂(Cl)₂(O₂CCH₂CH₂CO₂)₂];
Pt[(NH₃)₂(Cl)₂{O₂CC₆H₃(CO₂)₂}₂];
dichloro(2,2′-bipyridine-4,4′-dicarboxylato)platinum (II);
dichloro(2,2′-bipyridine-5,5′-dicarboxylato)platinum (II); and
Pt[(NH₃)₂(Cl)₂(ethylene diamine tetraacetate)₂].

In some embodiments, a stabilized NCP can be contacted with the cell. For example, in some embodiments, the NCP can comprise a core and an outer layer, wherein the core comprises a coordination polymer and the outer layer surrounds the core and comprises a group that shields the NCP from the aqueous environment, either partially or totally, for a period of time. Thus, in some embodiments, the NCP can be used in methods where controlled release of platinum metal complexes and/or other anticancer therapeutics is desired. The controlled release can relate to sustained release over a given period of time or to targeted release in the presence of a particular cell type or in the presence of particular conditions (e.g., pH, presence of a particular enzyme or biological reductant, etc). In some embodiments, the outer layer can comprise a metal oxide, an organic polymer (e.g., PVP), a silica-based polymer (e.g., SiO₂), a lipid bilayer, or a combination thereof.

In some embodiments, the NCP can comprise one or more of a photosensitizer, a radiosensitizer, a radionuclide, an imaging agent, a passivating agent, and a targeting agent. Thus, in some embodiments, the NCP can comprise a photosensitizer and the method can further comprise administering light to the cancer cell (e.g., laser light of a particular wavelength or wavelength range) during or after contacting the cell with the NCP. In some embodiments, the NCP can comprise a radiosensitizer, and the method can further comprise administering radiation or a source of radiation to the cancer cell during or following the contacting of the cell with the NCP. For example, the NCP can comprise both a radiosensitizer and a radionuclide in addition to the platinum metal complexes and/or small molecule anticancer drugs and administering the NCP to the cell provides both chemotherapeutic treatment and radiation therapy.

In some embodiments, the NCP can comprise a targeting agent that directs entry of the NCP or its components into the cell via receptor-mediated endocytosis. For example, in some embodiments, the cell can comprise an angiongenic cancer cell and/or a cancer cell known to express an integrin receptor on its surface, and the NCP can comprise a targeting agent comprising the ROD tripeptide motif, such as a targeting agent comprising c(RGDfK).

In some embodiments, the NCP can comprise one or more imaging agents. The imaging agents can include, but are not limited to, optical imaging agents, MRI agents, PET agents, SPECT agents and combinations thereof. Thus, in some embodiments, the method further comprises imaging the cancer cell or the sample comprising the cancer cell via one or more suitable imaging technique. The imaging can be performed prior to contacting the cell with the NCP, while contacting the cell with the NCP, after contacting the cell with the NCP, or any combination thereof. The imaging can be performed, for example, to show entry of the NCP into one or more cancer cells, to show the location of the cancer cells in a larger sample (e.g., a tissue, organ, or subject), or to provide evidence of reduction of proliferation of the cancer cells via administration with the NCP.

In some embodiments, the cancer cell is present in an in vitro sample (e.g., a cell culture, a cell culture extract, or an excised organ or tissue). In some embodiments, the cell is present in a subject.

In some embodiments, the presently disclosed subject matter provides a method of treating cancer in a subject in need of treatment thereof, the method comprising administering to the subject a nanoparticle comprising a coordination polymer, wherein the coordination polymer comprises a plurality of platinum metal complexes. By “treating cancer” is meant providing the nanoparticle in an effective amount to prevent proliferation of the cancer (e.g., to prevent an increase in the size of a tumor or the number of cancer cells present in a subject or to prevent metastasis of the cancer to additional locales (e.g., organs or tissues) in the body of the subject), to reduce the size of a tumor related to the cancer or to otherwise reduce the number of cancer cells present in the subject, or to relieve one or more symptoms related to the presence of the cancer in the subject.

The cancer to be treated according to the presently disclosed methods can include any cancer, such as, but not limited to a skin cancer, a connective tissue cancer, an esophogeal cancer, a breast cancer, a lung cancer, a stomach cancer, a pancreatic cancer, an ovarian cancer, a cervical cancer, a uterine cancer, an anogenital cancer, a kidney cancer, a bladder cancer, a colon cancer, a prostate cancer, a retinal cancer, a central nervous system cancer, or a lymphoid cancer. In some embodiments, the cancer is breast cancer or colon cancer.

In some embodiments, the subject is a mammal. In some embodiments, the subject is a human. In some embodiments, the nanoparticle is administered in a liposomal or microemulsion formulation. The nanoparticle can be administered via any suitable route. In some embodiments, the NCP is administered via an intravenous route or intratumoral route of administration.

The NCP can comprise a plurality of platinum (II) metal complexes, a plurality of platinum (IV) metal complexes, or a combination thereof. One or more of the platinum metal complexes can comprise:
a platinum metal atom;
at least one nonbridging ligand, wherein the at least one nonbridging ligand is bonded to the platinum metal atom through at least one coordination bond; and
at least one bridging ligand, wherein the at least one bridging ligand is bonded to the platinum metal atom through at least one coordination bond and comprises at least one linking moiety, wherein each of the at least one linking moiety is bonded to an additional metal atom via a coordination bond.

Suitable linking moieties include, but are not limited to, carboxylate, carboxylic acid, ester, carbamate, carbonate, amine (e.g., primary, secondary, tertiary, or aromatic amine), amide, hydroxyl, thiol, ether, thioether, phosphonate, and phosphate. Nonbridging ligands can be independently selected from the group including, but not limited to, NH₃, primary amines, secondary amines, tertiary amines, diamines, aromatic amines, halides, hydroxy-substituted alkyl, hydroxy-substituted aryl, esters, carboxylates, carboxylic acids, carbamates, amides, thiols, hydroxide, and combinations thereof.

In some embodiments, the bridging ligand can comprise at least two carboxylate groups. Bridging ligands can be either polymeric or nonpolymeric. In some embodiments, the NCP comprises only nonpolymeric bridging ligands.

The NCP can comprise one or more platinum metal complex(es) that comprise(s) two bridging ligands, where each of the two bridging ligands is bonded to the platinum metal atom through one coordination bond and comprises at least one linking moiety. Each of the two bridging ligands can be selected from a BDC or BTC. In some embodiments, each of the two bridging ligands can be independently selected from the group including but not limited to 1,4-benzene dicarboxylate; 1,3,5-benzene tricarboxylate; succinate; and ethylene diamine tetraacetate.

The NCP can comprise one or more platinum metal complex(es) that comprise(s) one bridging ligand, where the one bridging ligand is bonded to the platinum metal atom through two coordination bonds and comprises at least two linking moieties. In some embodiments, the bridging ligand is a bipyridine dicarboxylate, such as, but not limited to, 2,2′-bipyridine-5,5′-dicarboxylate and 2,2′-bipyridine-4,4′-dicarboxylate.

In some embodiments, the NCP comprises one or more platinum metal complex(es) that comprise(s) a bridging ligand where the bridging ligand is an nonplatinum anticancer drug or anticancer prodrug. Suitable nonplatinum anticancer drug bridging ligands include, but are not limited to, methotrexate, folic acid, leucovorin, vinblastine, vincristine, melphalan, imatinib, pemetrexed, vindesine, anastrozole, doxorubicin, cytarabine, azathioprine, letrozole and carboxylates thereof.

In some embodiments, the NCP can comprise at least one polymeric bridging ligand. Polymeric bridging ligands can comprise polymers such as, but not limited to, poly(silsesquioxane), poly(siloxane), poly(acrylate) and poly(acrylamide).

In some embodiments, the NCP is bimetallic and includes one or more additional nonplatinum metal atom in addition to the platinum metal atoms of the platinum metal complexes. The nonplatinum metal atoms can include nonplatinum transition metal atoms, lanthanide metal atoms, and actinide metal atoms. For example, the NCP can be a bimetallic coordination polymer comprising formulas such as Tbₓ[Pt(L)ₙ(BL)ₘ]_{y} or Znₓ[Pt(L)ₙ(BL)ₘ]_{y}, wherein each L is a nonbridging ligand and each BL is a bridging ligand.

In some embodiments, each of the plurality of platinum metal complexes in the NCP can be independently selected from the group including but not limited to:
Pt[(NH₃)₂(Cl)₂(O₂CCH₂CH₂CO₂)₂];
Pt[(NH₃)₂(Cl)₂{O₂CC₆H₃(CO₂)₂}₂];
dichloro(2,2′-bipyridine-4,4′-dicarboxylato)platinum (II);
dichloro(2,2′-bipyridine-5,5′-dicarboxylato)platinum (II); and
Pt[(NH₃)₂(Cl)₂(ethylene diamine tetraacetate)₂].

In some embodiments, a stabilized NCP can be administered to the subject. For example, in some embodiments, the NCP can comprise a core and an outer layer, wherein the core comprises a coordination polymer and the outer layer surrounds the core and comprises a group that shields the NCP from the aqueous environment in vivo following administration, either partially or totally, for a period of time. Thus, in some embodiments, the NCP can be used in methods where controlled release of platinum metal complexes and/or other anticancer therapeutics is desired. The controlled release can relate to sustained release over a given period of time or to targeted release in the presence of a particular cell type or in the presence of particular conditions (e.g., pH, presence of a particular enzyme or biological reductant, etc). In some embodiments, the outer layer can comprise a metal oxide, an organic polymer (e.g., PVP), a silica-based polymer (e.g., SiO₂), a lipid bilayer, or a combination thereof.

In some embodiments, the NCP further comprises one or more of a photosensitizer, a radiosensitizer, a radionuclide, a passivating agent, an imaging agent, and a targeting agent. Thus, in some embodiments, the NCP can comprise a photosensitizer and the method can further comprise administering an external stimulus (e.g., laser light of a particular wavelength or wavelength range) to the subject (e.g., to a tumor site within the subject) during or after administering the nanoparticle.

In some embodiments, the nanoparticle can comprise a radiosensitizer, and the method can further comprise administering radiation or a source of radiation (e.g., a radionuclide) to the subject during or following administering the nanoparticle. For example, the nanoparticle can comprise both a radiosensitizer and a radionuclide in addition to the platinum metal complexes and/or small molecule anticancer drugs and administering the nanoparticle to the cells provides both chemotherapeutic treatment and radiation therapy. In some embodiments, the radiation or radiation source comprises an external stimulus (e.g., X-ray radiation).

In some embodiments, the NCP can comprise a targeting agent that directs delivery of the nanoparticle to a particular type of cell, organ or tissue within the subject. For example, the NCP can comprise a targeting agent specific for cancer cells or for a particular type of cancer cells. In some embodiments, the NCP can comprise a targeting agent comprising the ROD tripeptide motif, such as a targeting agent comprising c(RGDfK).

In some embodiments, the NCP can comprise one or more imaging agents. The imaging agents can include, but are not limited to, optical imaging agents, MRI agents, PET agents, SPECT agents and combinations thereof. Thus, in some embodiments, the method can further comprise imaging the comprising imaging delivery of the nanoparticle in one or more cells, tissues or organs in the subject following administration of the nanoparticle via one or more suitable imaging technique. The imaging can be performed prior to, during, or after administration of the nanoparticle, or in any combination thereof. The imaging can be performed, for example, to show entry of the nanoparticles into one or more cancer cells, to show the location and/or size of the cancer, or to provide evidence that administration of the nanoparticle provides effective treatment of the cancer.

### XIV. Synthesis of Nanoscale Coordination Polymers

The NCPs can be prepared via any suitable method. Typically, the NCPs can be synthesized either via precipitation methods involving initiator solvents (also referred to as “poor” solvents) or via microemulsion techniques. For example, the precipitation methods comprise providing solutions of the components of the coordination polymers (e.g., the platinum metal complexes, bridging ligands, and/or additional metal atoms or components) in solvents in which the components are soluble (i.e., readily dissolve to provide a clear solution). Addition of a solvent or solvent mixture (i.e., the initiator or “poor” solvent) in which the components are not as soluble leads to polymerization of the components and precipitation of the resultant nanoparticles. In microemulsion techniques, the components of the NCPs are provided in one or more microemulsion solutions. Contacting and/or stirring the microemulsion solutions leads to polymerization of the components and formation of the NCPs.

Accordingly, in some embodiments, the presently disclosed subject matter provides a method of synthesizing a nanoparticle comprising a coordination polymer comprising a plurality of platinum metal complexes, wherein the method comprises:
providing a solution comprising a first solvent, at least one bridging ligand precursor, and a plurality of platinum diaqua complexes selected from the group consisting of platinum (II) diaqua complexes, platinum (IV) diaqua complexes, and mixtures thereof; and
adding a second solvent to the solution to precipitate the nanoparticle.

In some embodiments, the method further comprises adjusting the pH of the solution prior to adding the second solvent. For example, when the first solvent comprises water, addition of an aqueous solution of a base (e.g., NaOH, KOH, LION, or sodium carbonate) can increase the pH of the solution if desired, while addition of an aqueous solution of an acid (e.g., HCl or sulfuric acid) can lower the pH when desired. Adjusting the pH can be performed, for instance, to increase the solubility of a bridging ligand precursor or of diaqua complexes in the solution.

The first solvent can be any solvent or solvent mixture which, when provided in a suitable volume, will dissolve an amount of bridging ligand precursor and diaqua complex sufficient for preparing a desired amount of NCP. In some embodiments, the first solvent can comprises water, dimethyl sulfoxide (DMSO), or a combination thereof.

In some embodiments, the at least one bridging ligand precursor comprises a nonpolymeric molecule. In some embodiments, the at least one bridiging moiety can be a molecule such as, but not limited to, a benzene dicarboxylic acid, a benzene dicarboxylate, a carboxylate-substituted styrene, a carboxylate-substituted silyl ether, a bipyridine dicarboxylic acid, a bipyridine dicarboxylate, a dicarboxylic anhydride, a diacyl dichloride, and a nonplatinum anticancer drug. Suitable nonplatinum anticancer drugs include, but are not limited to, methotrexate, folic acid, leucovorin, vinblastine, vincristine, melphalan, imatinib, pemetrexed, vindesine, anastrozole, doxorubicin, cytarabine, azathioprine, letrozole, and carboxylates thereof.

The second solvent should be a solvent that when mixed into the solution, decreases the solubility of one or more of the bridging ligand precursor, the platinum diaqua complexes, or the NCPs prepared therefrom. In some embodiments, the second solvent is selected from the group including but not limited to acetone, an alcohol, ether, acetonitrile, and mixtures thereof.

The solution can comprise one or more additional components. For example, when bimetallic NCPs are being prepared, the solution can further comprise metal compounds that contain nonplatinum metal atoms. The nonplatinum metal atoms can be nonplatinum transition metal atoms, lanthanide metal atoms, actinide metal atoms, or combinations thereof. In some embodiments, the nonplatinum metal atom is Tb³⁺ or Zn²⁺. Thus, for example, the solution can comprise a Tb-containing compound, such as, but not limited to, TbCl₃.

In some embodiments, the bridging ligand precursor is polymeric or comprises a polymerizable monomer. Suitable polymerizable monomers can include, but are not limited to acrylic acid, acrylamide, and silyl ethers.

In some embodiments, the solution can further comprise an additional component such as a radionuclide, an imaging agent, a photosensitizer, and a radiosensitizer, and adding the second solvent co-precipitates the additional component, thereby incorporating the additional component into the nanoparticle (e.g., as an additional metal atom in the coordination polymer if the additional component comprises a radionuclide, covalently bound to components of the coordination polymer, or physically sequestered in pores within the coordination polymer).

Alternatively, the NCPs can be prepared from microemulsions. Microemulsions, particularly, water-in-oil, or reverse, microemulsions have been used to synthesize a variety of nanophase materials such as organic polymers, semiconductor nanoparticles (see Xu and Akins, Material. Letters, 58, 2623 (2004)), metal oxides, and nanocrystals consisting of cyanide-bridged transition metal ions. See Vaucher et al. Angew. Chem. Int. Ed., 39, 1793 (2000); Vaucher et al., Nano Lett., 2, 225 (2002); Uemura and Kitagawa, J. Am. Chem. Soc., 125, 7814 (2003); Catala et al., Adv. Mater., 15, 826 (2003); and Yamada et al., J. Am. Chem. Soc., 126, 9482 (2004). Reverse microemulsions comprise nanometer scale water droplets stabilized in an organic phase by a surfactant, which can be anionic, cationic, or neutral in charge. Numerous reports on the physical properties of microemulsion systems suggest the water to surfactant ratio, referred to as the W value (i.e., [H₂O]/[surfactant]), largely dictates the size of the reverse micelle, which is just one of many tunable properties microemulsions offer. See Wong et al., J. Am. Chem. Soc., 98, 2391 (1976); White et al., Langmuir, 21, 2721 (2005); Giustini et al., J. Phys. Chem., 100, 3190 (1996); and Kumar and Mittal, eds., Handbook of Microemulsion Science and Technology; New York: Marcel Decker, 1999. For a description of the use of microemulsions in preparing silica-coated nanoparticles, see U.S. Patent Application Publication No. 20060228554, which is incorporated herein by reference in its entirety.

In some embodiments, the presently disclosed subject matter provides a method of synthesizing a nanoparticle comprising a coordination polymer for use as an anticancer therapeutic agent or a multi-modal agent. In particular, the presently disclosed synthesis methods involve the use of microemulsions in preparing NCPs. The microemulsion can be water-in-oil (i.e., reverse micelles or water droplets dispersed in oil), oil-in-water (i.e., micelles or oil droplets dispersed in water), or a bi-continuous system containing comparable amounts of two immiscible fluids. In some cases, microemulsions can be made by mixing together two non-aqueous liquids of differing polarity with negligible mutual solubility.

The immiscible liquids that can be used to make the microemulsion typically include a relatively polar (i.e., hydrophobic) liquid and a relative non-polar (i.e., hydrophillic) liquid. While a large variety of polar/non-polar liquid mixtures can be used to form a microemulsion useful in the invention, the choice of particular liquids utilized can depend on the type of nanoparticles being made. Upon a review of the instant disclosure, a skilled artisan can select specific liquids for particular applications by adapting known methods of making microemulsions for use in the presently disclosed methods. In many embodiments, the relatively polar liquid is water, although other polar liquids might also be useful. Water is useful because it is inexpensive, readily available, non-toxic, easy to handle and store, compatible with a large number of different precipitation reactions, and immiscible in a large number of non-polar solvents. Examples of suitable non-polar liquids include alkanes (e.g., any liquid form of hexane, heptane, octane, nonane, decane, undecane, dodecane, etc.), cycloalkanes (e.g., cyclopentane, cyclohexane, etc.), aromatic hydrocarbons (e.g., benzene, toluene, etc.), and mixtures of the foregoing (e.g., petroleum and petroleum derivatives). In general, any such non-polar liquid can be used as long as it is compatible with the other components used to form the microemulsion and does not interfere with any precipitation reaction used to isolate the particles after their preparation.

Generally, at least one surfactant is needed to form a microemulsion. Surfactants are surface active agents that thermodynamically stabilize the very small dispersed micelles or reverse micelles in microemulsions. Typically, surfactants possess an amphipathic structure that allows them to form films with very low interfacial tension between the oily and aqueous phases. Thus, any substance that reduces surface tension at the interface of the relatively polar and relatively non-polar liquids and is compatible with other aspects of the presently disclosed subject matter can be used to form the microemulsion used to make nanoparticles. The choice of a surfactant can depend on the particular liquids utilized and on the type of nanoparticles being made. Specific surfactants suitable for particular applications can be selected from known methods of making microemulsions or known characteristics of surfactants. For example, non-ionic surfactants are generally preferred when an ionic reactant is used in the microemulsion process and an ionic detergent would bind to or otherwise interfere with the ionic reactant.

Numerous suitable surfactants are known. A nonexhaustive list includes soaps such as potassium oleate, sodium oleate, etc.; anionic detergents such as sodium cholate, sodium caprylate, etc.; cationic detergents such as cetylpyridynium chloride, alkyltrimethylammonium bromides, benzalkonium chloride, cetyldimethylethylammonium bromide, etc; zwitterionic detergents such as N-alkyl-N,N-dimethylammonio-1-propanesulfonates and CHAPS; and non-ionic detergents such as polyoxyethylene esters, and various tritons (e.g., (TRITON™-X100, TRITON™-X114); etc.

The concentration of surfactant used can depend on many factors including the particular surfactant selected, liquids used, and the type of nanoparticles to be made. Suitable concentrations can be determined empirically; i.e., by trying different concentrations of surfactant until the concentration that performs best in a particular application is found. Ranges of suitable concentrations can also be determined from known critical micelle concentrations.

In some embodiments, the presently disclosed subject matter provides a method of preparing a nanoscale coordination polymer comprising a plurality of platinum metal complexes where the method comprises:
providing a first mixture comprising a microemulsion system comprising water, an organic solvent, a surfactant, and a co-surfactant;
adding to the first mixture an aqueous solution comprising a platinum metal complex, thereby forming a second mixture, wherein the platinum metal complex comprises a platinum metal atom, one or more nonbridging ligands, and at least one ligand bound to the platinum metal atom by at least one coordination bond and comprising at least one prelinking moiety, wherein the at least one prelinking moiety is a group that can form a coordination bond with an additional metal atom; and
stirring the second mixture for a period of time, thereby synthesizing the nanoparticle.

In some embodiments, the presently disclosed subject matter provides a method of preparing a bimetallic nanoscale coordination polymer using microemulsion techniques. For example, in some embodiments, the presently disclosed subject matter provides a method of synthesizing a nanoparticle comprising a coordination polymer comprising a plurality of platinum metal complexes, the method comprising:
providing a first mixture comprising a microemulsion system comprising water, an organic solvent, a surfactant, and a co-surfactant;
adding to the first mixture an aqueous solution comprising a platinum metal complex, thereby forming a second mixture, wherein the platinum metal complex comprises a platinum metal atom, one or more nonbridging ligands, and at least one ligand bound to the platinum metal atom by at least one coordination bond and comprising at least one prelinking moiety, wherein the at least one prelinking moiety is a group that can form a coordination bond with an additional metal atom;
stirring the second mixture until the second mixture is visably clear;
providing a third mixture comprising a microemulsion system comprising water, an organic solvent, a surfactant, and a co-surfactant;
adding to the third mixture an aqueous solution comprising a nonplatinum metal compound, thereby forming a fourth mixture;
stirring the fourth mixture until the fourth mixture is visably clear;
adding the fourth mixture and the second mixture to form a fifth mixture; and
stirring the fifth mixture for a period of time, thereby synthesizing the nanoparticle.

The nonplatinum metal compound can comprise a nonplatinum metal atom which can be a nonplatinum transition metal atom, a lanthanide metal atom, an actinide metal atom or combinations thereof. For example, the nonplatinum metal atom can be a paramagentic metal atom useful in MRI imaging. The nonplatinum metal atom could also be a metal atom useful in a PET or SPECT imaging agent. In some embodiments, the nonplatinum metal atom is Tb³⁺ or Zn²⁺. In some embodiments, the nonplatinum metal compound is TbCl₃.

In some embodiments, the mixing comprises stirring (e.g., using a magnetic stirrer or a mechanical stirrer). Mixing can also refer to sonication or to manual or mechanical shaking, or to any combination thereof.

In some embodiments, the surfactant is a non-ionic surfactant. In some embodiments, the surfactant is TRITON™-X100. In some embodiments, the co-surfactant is 1-hexanol. In some embodiments, the molar ratio of TRITON™-X100 to 1-hexanol ranges between about 1 and about 5.

When preparing nanoparticles comprising coordination polymers, useful water to surfactant ratios (i.e., W, the ratio of [water]/[surfactant]) for the third mixture (i.e., after the addition of the polymerization agent, which can contribute to the water content of the mixture if dissolved in an aqueous carrier) range from about 10 to about 25 (i.e., 10, 11, 12, 13, 14, 15, 16, 17, 18, 19, 20, 21, 22, 23, 24, or 25). As described hereinbelow, in the examples, varying W can lead to variations in the size of the resulting nanoparticles.

The presently disclosed subject matter also provides microemulsion methods of preparing nanoscale coordination polymers comprising polymeric bridging ligands. Thus, in some embodiments, the presently disclosed subject matter provides a method of synthesizing a nanoparticle comprising a coordination polymer comprising a plurality of platinum metal complexes, wherein the method comprises providing a microemulsion system comprising water; an organic solvent; a surfactant; a co-surfactant; a polymerizable monomer; and a platinum metal complex, wherein the platinum metal complex comprises a platinum metal atom, one or more nonbridging ligands, and at least one ligand bonded to the platinum metal atom by at least one coordination bond and comprising at least one prelinking moiety, where the at least one prelinking moiety is a moiety that can react with the polymerizable monomer. In some embodiments, a polymerization agent can be added to the microemulsion system to initiate polymerization of the polymerizable monomer.

Suitable prelinking moieties include, but are not limited to, alkyl halides, acyl halides, silyl ethers, alkenes, alkynes, carboxylic acids, amines, esters, anhydrides, and isocyanates. Suitable polymerizable monomers include, but are not limited to, silyl ethers (e.g., TEOS), acrylic acid, and acrylamide. When TEOS is the polymerizable monomer, the polymerization agent can be aqueous ammonia. Other suitable polymerization agents include aqueous hydroxide (e.g., NaOH) or hydrazine. When acrylic acid (or another acrylic monomer) is the polymerizable monomer, a suitable polymerization agent is tetramethylethane diamine (TMEDA). A suitable surfactant is cetyltimethyl ammonium bromide (CTAB). The microemulsion water to surfactant ratio can range from about 5 to about 15 (i.e., 5, 6, 7, 8, 9, 10, 11, 12, 13, 14, or 15).

NCPs prepared by precipitation or microemulsion techniques can be isolated from the solutions used in their synthesis via any suitable technique (i.e., filtration, decanting, lyophilization, evaporation or vacuum evaporation of the solutions). In some embodiments, the isolation of the nanoparticles can be done by centrifugation.

In some embodiments, the method of synthesizing the nanoparticle comprises further steps to graft additional components onto the surface of the nanoparticle or to coat the nanoparticle with an outer layer. For example, the methods can further comprise coating the nanoparticle with one or more of a metal oxide, a lipid bilayer, an organic polymer, a silica-based polymer, and combinations thereof. Silica-based polymers can be coated onto the nanoparticles by sol-gel techniques known in the art. Accordingly, in some embodiments, the method of synthesizing the nanoparticle further comprises grafting onto the surface of the nanoparticle one or more of a photosensitizer, a radiosensitizer, a radionuclide, an imaging agent, a passivating agent, and a targeting agent.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Materials and Methods

All reagents and solvents were purchased from commercial sources as used without further purification unless otherwise stated. Thermogravimetric analysis (TGA) was performed using a Shimadzu TGA-50 (Shimadzu Corp., Kyoto, Japan) equipped with a platinum pan and heated at a rate of 3° C./min under air. Powder X-ray diffraction (PXRD) patterns were collected on a Bruker SMART APEX II diffractometer (Bruker AXS, Inc., Madison, Wis., United States of America) using Cu radiation. The PXRD patterns were processed with the APEX 2 package using phase ID plugin. A Hitachi 4700 field emission scanning electron microscope (SEM; Hitachi Ltd., Tokyo, Japan) and a JEM 100CX-1I transmission electron microscope (TEM; JEOL Ltd., Tokyo, Japan) were used to determine particle size and morphology. SEM images of the nanoparticles were taken on glass substrate. A Cressington 108 Auto Sputter Coater (Cressington Scientific Instruments, Ltd., Watford, United Kingdom) equipped with an Au/Pd (80/20) target and MTM-10 thickness monitor was used to coat the sample with a conductive layer before taking SEM images. TEM micrographs were obtained on carbon-coated copper grids. A Beckman Coulter N5 Submicron Particle Size Analyzer (Beckman Coulter, Fullerton, Calif., United States of America) was used to determine the sample's hydrodynamic diameter and polydispersity. A Varian 820-MS Inductively Coupled Plasma-Mass Spectrometer (ICP-MS; Varian, Inc., Palo Alto, Calif., United States of America) was used to determine metal concentration.

### Example 1

### Platinum (II) Nanoscale Coordination Polymers (NCPs)

### Example 1.1

### cis-Diaquadiammine Platinum(II) Dinitrate, [Pt(NH₃)₂(H₂O)₂](NO₃)₂

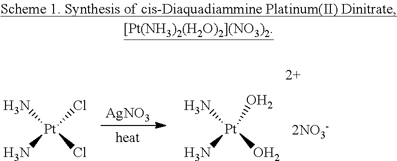

As shown above in Scheme 1, a mixture of cisplatin (300.06 mg, 1.0 mmol) and AgNO₃ (332.9 mg, 1.96 mmol) in pH=3 HNO₃ (10 mL) was magnetically stirred at 70° C. for 20 h in the dark. After cooling the reaction mixture to room temperature the AgCl byproduct was removed via filtration and washed with excess H₂O. The resultant solution was passed through a 0.45 μm filter and the solvent was removed on the rotary evaporator. A yellow crystalline material was isolated in 70% yield, which could be dissolved in water with mild heating.

### Example 1.2

### Dichloro(1R,2R-cyclohexanediammine)-platinum(II), Pt(R,R-DACH)Cl₂

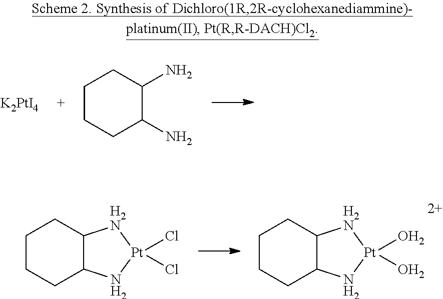

As shown in Scheme 2, above, a solution of K₂PtCl₄ (1.66 g, 4.00 mmol) and 1R,2R-cyclohexanediammine (0.460 mg, 4.00 mmol) in H₂O (20 mL) was magnetically stirred at room temperature for 20 h in the dark. The pale yellow powder was collected by vacuum filtration and washed successively with H₂O, EtOH, and acetone. Yield: 87.6%.

### Example 1.3

### Synthesis of Diaqua(1R,2R-cyclohexanediammine)platinum(II), [Pt(R,R-DACH)(H₂O)₂]²⁺

A mixture of Pt(R,R-DACH)Cl₂ (0.152 g, 0.40 mmol) and AgNO₃ (0.136 g, 0.798 mmol) in pH 3 HNO₃ (10 mL) was magnetically stirred at 70° C. for 24 h in the dark. After cooling the reaction mixture to room temperature the AgCl byproduct was removed via filtration and washed with excess H₂O. The resultant solution was passed through a 0.2 μm filter, concentrated on the rotary evaporator, and diluted to 2 mL with H₂O in a volumetric flask to yield an approximately 0.20 M solution of the cationic diaqua complex. The Pt(II) concentration was confirmed by direct current plasma (DCP) atomic emission spectroscopy.

### Example 1.4

### Preparation of Pt(II) NCPs Via the Addition of an Initiator/Poor Solvent

A precursor solution was prepared by mixing the platinum(II) diaqua complex and the bridging ligand in distilled water to reach a final solute concentration on the order of 10.3 M. At this time the pH of the solution could be adjusted via the addition of dilute NaOH or HCl. A poor solvent was rapidly added to the precursor solution to initiate precipitation of the desired product. The nanoparticles were isolated via centrifugation and washed with ethanol before redispersement in ethanol via sonication.

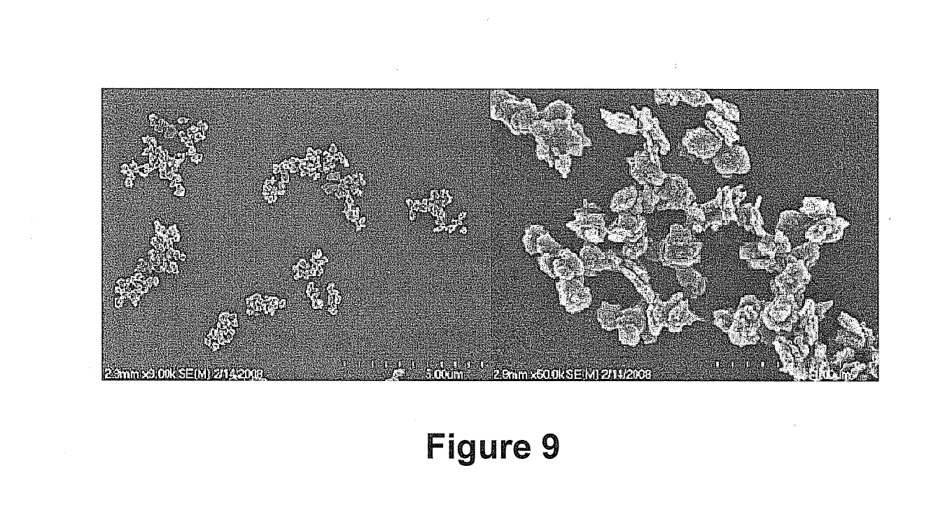

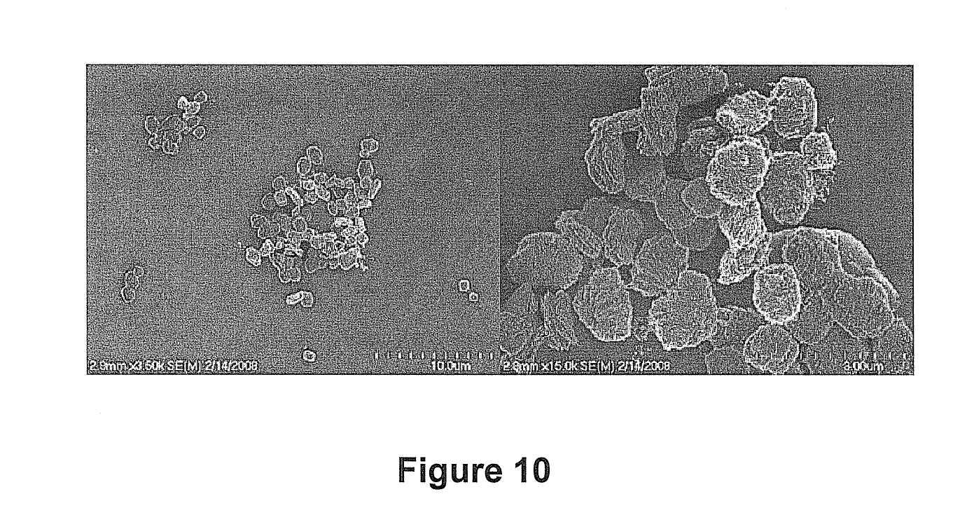

For example, the pH of a mixture of [cis Pt(NH₃)₂(OH₂)₂]²⁺ (1.0 μmol) and benzene dicarboxylate (BDC, 1.0 μmol) in 200.0 μL distilled water was adjusted to 5.5 with dilute NaOH. Acetone was rapidly added to the magnetically stirred precursor solution, which resulted in the formation of a clear solution that was bluish-white in color. The resulting mixture was magnetically stirred in the dark for an additional 1 h before isolating the product via centrifugation, washing with ethanol, and redispersing in ethanol via sonication. FIG. 9 shows the SEM micrographs of cis-Pt(NH₃)₂(BDC) nanoclusters synthesized via the rapid addition of acetone to a precursor (aqueous) solution of the components. FIG. 10 shows the SEM micrographs of cis-Pt(NH₃)₂(BDC) nanoclusters synthesized via the rapid addition of an acetone:ethanol mixture (1:1 v/v) to a precursor (aqueous) solution of the components.

### Example 2

### Bimetallic Nanoscale Coordination Polymers

### Example 2.1.1

### Synthesis of c,c,t-Pt(NH₃)₂Cl₂(O₂CCH₂CH₂CO₂H)₂ (DCSP)

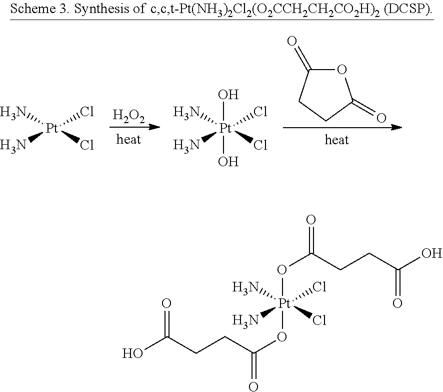

As shown in Scheme 3, a mixture of cis-Pt(NH₃)₂Cl₂ (i.e., cisplatin, 2.00 g, 6.67 mmol) and H₂O₂ (30 wt %, 11.37 mL, 100.0 mmol) in H₂O (90 mL, pH 7) was heated at 70° C. with vigorous magnetic stirring for 5 h in the dark. The heat was removed and stirring was continued overnight. After concentrating the mixture to about 10 mL, the product was allowed to precipitate at 4° C. over several hours. The product was collected via vacuum filtration, washed with ice cold H₂O, ethanol, and diethyl ether, and vacuum dried. The dihydroxide (c,c,t-Pt(NH₃)₂Cl₂(OH)₂ was obtained as a bright yellow powder in 85.8% yield (1.911 g).

As further shown in Scheme 3, a mixture of c,c,t-Pt(NH₃)₂Cl₂(OH)₂ (334 mg, 1.00 mmol) and succinic anhydride (400 mg, 4.00 mmol) in dimethyl sulfoxide (DMSO; 1.00 mL) was heated at 70° C. for 20 h with magnetic stirring. The solvent was subsequently removed under high vacuum at 70° C. The product (DCSP) was recrystallized from acetone at −20° C., isolated via vacuum filtration, and washed with ice cold acetone to yield a pale yellow powder. Yield: 96%.

### Example 2.1.2

### Synthesis of c,c,t-Pt(NH₃)₂Cl₂(BTC)₂

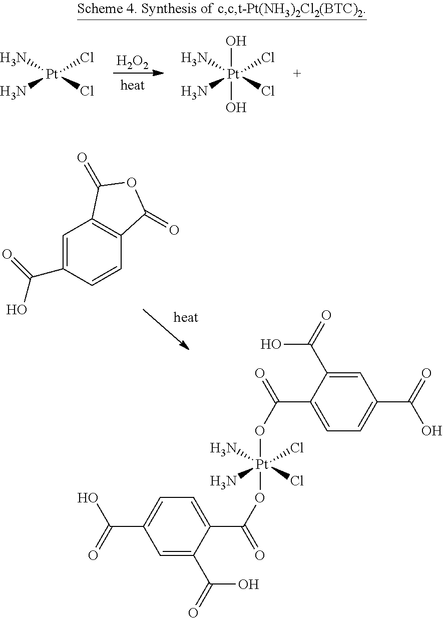

As shown in Scheme 4, a mixture of c,c,t-Pt(NH₃)₂Cl₂(OH)₂ (83.5 mg, 0.25 mmol) and benzene tricarboxylic anhydride (192.0 mg, 1.00 mmol) in dimethyl sulfoxide (DMSO; 0.50 mL) was heated at 70° C. under argon gas for 20 h with magnetic stirring. The solvent was subsequently removed under high vacuum at 70° C.

### Example 2.1.3

### Synthesis of 2,2′-bipyridine-4,4′-dicarboxylic acid

A mixture of 4,4′-dimethyl-2,2′-bipyridine and KMnO₄ in water (500 mL) was heated at 70° C. for 24 h with magnetic stirring. The brown precipitate was removed by filtration and washed multiple times with aqueous NaOH (1 M). The combined water fractions were extracted with CHCl₃ to remove unreacted starting material and subsequently neutralized with aqueous HCl (2 M). After concentrating the solution to a volume of approximately 300 mL, the solution was acidified further to a pH of 6 to precipitate the product. The light blue product was collected by centrifugation, washed with ethanol, and dried in vacuo.

### Example 2.1.4

### Synthesis of Dichloro(2,2′-bipyridine-4,4′-dicarboxylato)platinum(II)

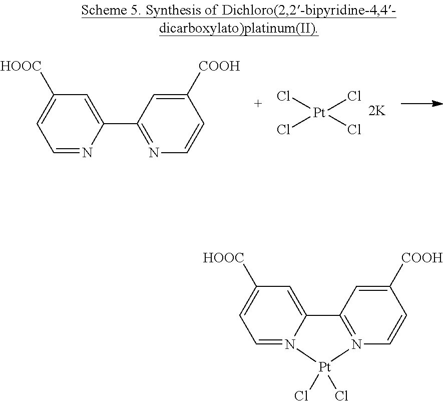

As shown in Scheme 5, an aqueous solution of aqueous KOH (0.10 M) was added dropwise to a mixture of 2,2′-bipyridine-4,4′-dicarboxylic acid (122 mg, 0.50 mmol) in water (10 mL) to reach a pH of about 8 and dissolve the ligand. To the solution was added K₂PtCl₄ (200 mg, 0.50 mmol) and KCl (150 mg), and it was subsequently heated at 70° C. for 20 h. The resulting yellow-orange mixture was acidified to a pH of 3-4 with dilute HCl. The product was collected by centrifugation, washed with water, and dried in vacuo.

### Example 2.1.5

### Synthesis of 2,2′-Bipyridine-5,5′-dicarboxylic acid

A mixture of 5,5′-dimethyl-2,2′-bipyridine and KMnO₄ in water (500 mL) was heated at 70° C. for 24 h with magnetic stirring. The brown precipitate was removed by filtration and washed multiple times with aqueous NaOH (1 M). The combined water fractions were extracted with CHCl₃ to remove unreacted starting material and subsequently neutralized with aqueous HCl (2 M). After concentrating the solution to a volume of approximately 300 mL, the solution was acidified further to a pH of 6 to precipitate the product. The light blue product was collected by centrifugation, washed with ethanol, and dried in vacuo.

### Example 2.1.6

### Synthesis of Dichloro(2,2′-bipyridine-5,5′-dicarboxylato)platinum(II)

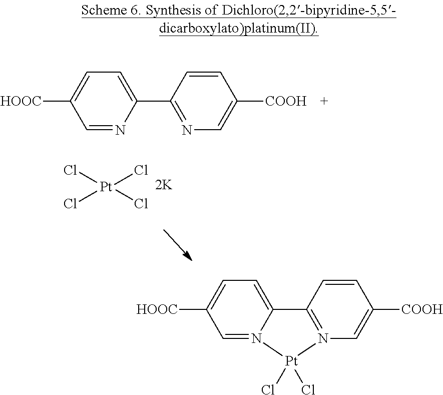

An aqueous solution of KOH (0.10 M) was added dropwise to a mixture of 2,2′bipyridine-5,5′-dicarboxylic acid (122 mg, 0.50 mmol) in water (10 mL) to reach a pH of −8 and dissolve the ligand. To the solution was added K₂PtCl₄ (200 mg, 0.50 mmol) and KCl (150 mg), and it was subsequently heated at 70° C. for 20 h. The resulting yellow-orange mixture was acidified to a pH of 3-4 with dilute HCl. The product was collected by centrifugation, washed with water, and dried in vacuo.

### Example 2.2.1

### Microemulsion Synthesis of Tbₓ[c,c,t-Pt(NH₃)₂Cl₂(O₂CCH₂CH₂CO₂)₂]_{y} coordination polymers

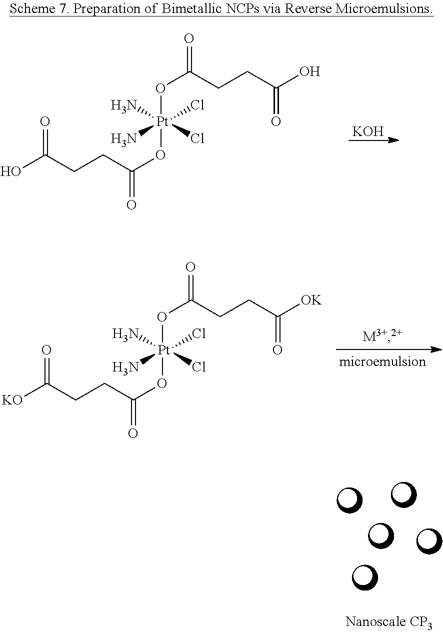

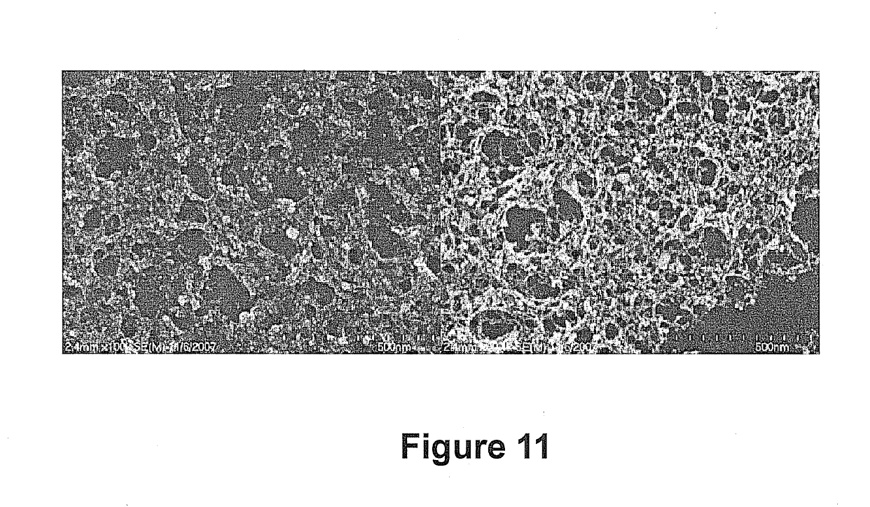

Nanometer-scale lanthanide-based coordination polymers with Pt(IV)-containing bridging complexes were prepared using a cationic cetyltrimethylammonium bromide (CTAB)/1-hexanol/iso-octane/H₂O microemulsion system. Briefly, a round bottom flask was charged with CTAB and a particular volume of 0.50 M 1-hexanol/isooctane solution to yield a milky white mixture with a CTAB concentration of 0.050 M. An aliquot of an aqueous solution of K₂-[c,c,t-Pt(NH₃)₂Cl₂(O₂CCH₂CH₂CO₂)₂] was then added to the above mixture corresponding to a particular W (water to surfactant molar ratio). A separate microemulsion was prepared with an equivalent volume of an aqueous solution of TbCl₃. After magnetically stirring the separate microemulsions until visibly clear, they were combined and stirred for an additional period of time before functionalization with polyvinylpyrrolidone (PVP) or isolation by means of centrifugation. FIG. 11 shows the SEM micrographs of Tbₓ[c,c,t-Pt(NH₃)₂Cl₂(O₂CCH₂CH₂CO₂)₂]_{y} nanoparticles prepared with a cationic microemulsions with W 15 or 20.

### Example 2.2.2

### Microemulsion Synthesis of Znₓ[c,c,t-Pt(NH₃)₂Cl₂(O₂CCH₂CH₂CO₂)₂]_{y} Coordination Polymers

Nanometer-scale coordination polymers with Pt(IV)-containing bridging complexes were prepared using a cationic cetyltrimethylammonium bromide (CTAB)/1-hexanol/iso-octane/H₂O microemulsion system. Briefly, a round bottom flask was charged with CTAB and a particular volume of 0.50 M 1hexanol in isooctane solution to yield a milky white mixture with a CTAB concentration of 0.05 M. An aliquot of an aqueous solution of K₂-[c,c,t-Pt(NH₃)₂Cl₂(O₂CCH₂CH₂CO₂)₂] was then added to the above mixture corresponding to a particular W (water to surfactant molar ratio). A separate microemulsion was prepared with an equivalent volume of an aqueous solution of ZnCl₂. After magnetically stirring the separate microemulsions until visibly clear, they were combined and stirred for an additional period of time before functionalization with polyvinylpyrrolidone (PVP) or isolation by centrifugation.

### Example 2.3.1

### Rapid Precipitation of Tbₓ[c,c,t-Pt(NH₃)₂Cl₂(O₂CCH₂CH₂CO₂)₂]_{y} Nanoscale Coordination Polymers

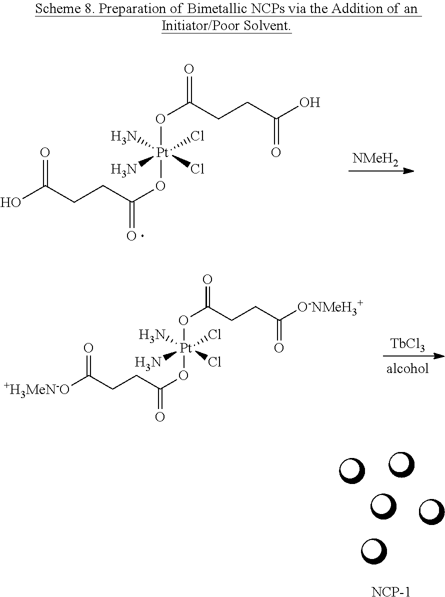

A precursor solution was prepared by mixing the dimethylammonium salt of the platinum complex and the metal (M²⁺ or M³⁺) in distilled water to reach a final solute concentration on the order of 10.2 M. At this time the pH of the solution could be adjusted via the addition of dilute NaOH or HCl. An initiator solvent was rapidly added to the precursor solution to initiate precipitation of the desired product. The nanoparticles were isolated via centrifugation and washed with ethanol before redispersement in ethanol via sonication.

For example, as shown in Scheme 8, a mixture of [c,c,t-Pt(NH₃)₂Cl₂(succ)₂].2N(CH₃)H₂ (i.e., the dimethylammonium salt of DSCP; 0.50 mmol) and TbCl₃ (0.75 mmol) in 50 mL distilled water was prepared in a 600 mL beaker. The pH of the solution was subsequently adjusted to 5.5 with dilute NaOH. Methanol was rapidly added to the magnetically stirred precursor solution, which resulted in the formation of a clear solution bluish-white in color. The resulting mixture was magnetically stirred in the dark for an additional 1 h before isolating the product via centrifugation, washing with methanol and ethanol, and redispersing in ethanol via sonication. Yield: 235 mg (73% isolated based on DSCP). The NCPs exhibited a spherical morphology and were structurally amorphous, yielding no PXRD peaks that would indicate a crystalline phase.

The composition of the particles was deduced from ICP-MS measurements and TGA data. ICP-MS measurements gave an approximate Tb:Pt molar ratio of 2:3, which is expected for the charge balanced formula Tb₂(DSCP)₃(H₂O)ₓ. Using this data, the number of water molecules was determined from TGA data. There was an approximately 9.2% weight loss for the water species and an approximately 36.0% weight loss for the organic species. The sum MW of the organic species (i.e., 2 NH₃ and 2 succinate groups) for DSCP was determined to be approximately 268. By taking into account the loss of 1.5 DSCP for every Tb atom in the formula, the approximate TGA formula weight of 1115 (268×1.5/0.36) was determined. All the TGA species were subtracted from the approximate TGA weight: 1115−158.9 (Tb)−24 (1.5 oxygen, TbO_{1.5} forms as a result of burning in air)−1.5×534 (1.5 DSCP)−24 (1.5 oxygen, to form PtOCl₂)=approximately 107. This number divided by the MW for water gave 6 water molecules per Tb, and thus an empirical formula of Tb₂(DCSP)₃(H₂O)₁₂ for NCP-1. This formula was not unexpected as it (a) results in a charge balanced material and (b) the six water molecules fill the remaining sites of coordination on the Tb metal ion. The consistent Tb/Pt molar ration of the nanoparticles prepared according to the presently disclosed methods further suggest that the NCPs are Tb-terminated.

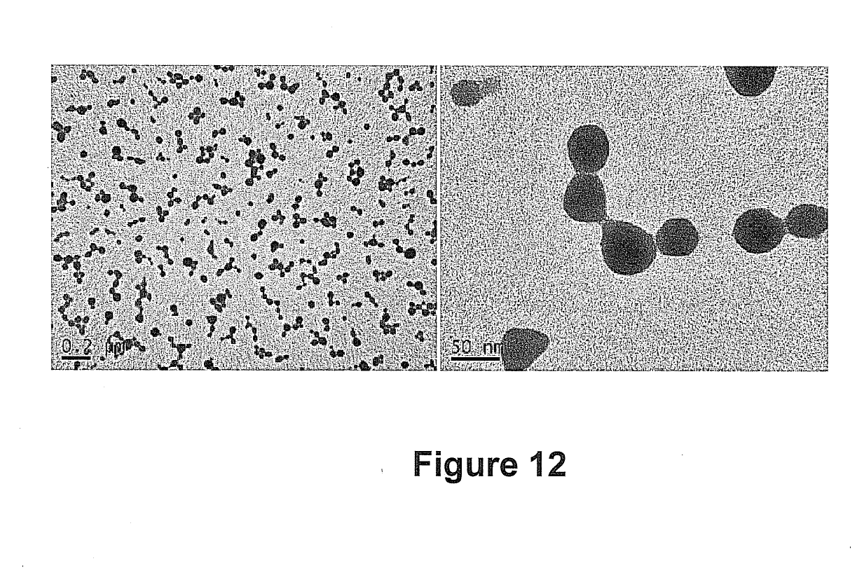

FIG. 12 shows the SEM micrographs of Tbₓ[c,c,t-Pt(NH₃)₂Cl₂(O₂CCH₂CH₂CO₂)₂]_{y} (i.e., NCP-1) nanoparticles synthesized via the rapid addition of methanol to a precursor (aqueous) solution of the components. DLS measurements gave a diameter of 58.3±11.3 nm for the NCP-1 particles. The NCPs were stable and readily dispersible in most organic solvents. NCP formation was reversible if excess water was added to the reaction mixture or to the isolated particles.

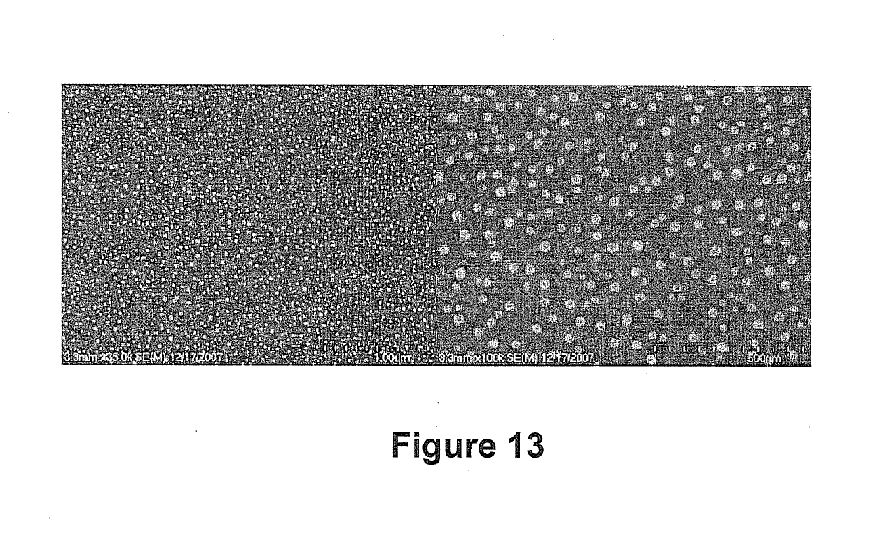

Alternatively, the Tbₓ[c,c,t-Pt(NH₃)₂Cl₂(O₂CCH₂CH₂CO₂)₂]_{y} nanoparticles can be synthesized by rapid addition of a 1:1 ethanol/methanol solution of an aqueous solution of [c,c,t-Pt(NH₃)₂Cl₂(succ)₂].2N(CH₃)H₂ and TbCl₃. FIG. 13 shows SEM images of these Tbₓ[c,c,t-Pt(NH₃)₂Cl₂(O₂CCH₂CH₂CO₂)₂]_{y} nanoparticles.

### Example 2.3.2

### Rapid Precipitation of M(II)ₓ[c,c,t-Pt(NH₃)₂Cl₂(O₂CCH₂CH₂CO₂)₂]_{y} Nanoscale Coordination Polymers

A precursor solution was prepared by mixing the dimethylammonium salt of the platinum complex and the second metal in distilled water to reach a final solute concentration on the order of 10.3 M. At this time the pH of the solution could be adjusted via the addition of dilute NaOH or HCl. An initiator solvent was rapidly added to the precursor solution to initiate precipitation of the desired product. The nanoparticles were isolated via centrifugation and washed with ethanol before redispersement in ethanol via sonication.

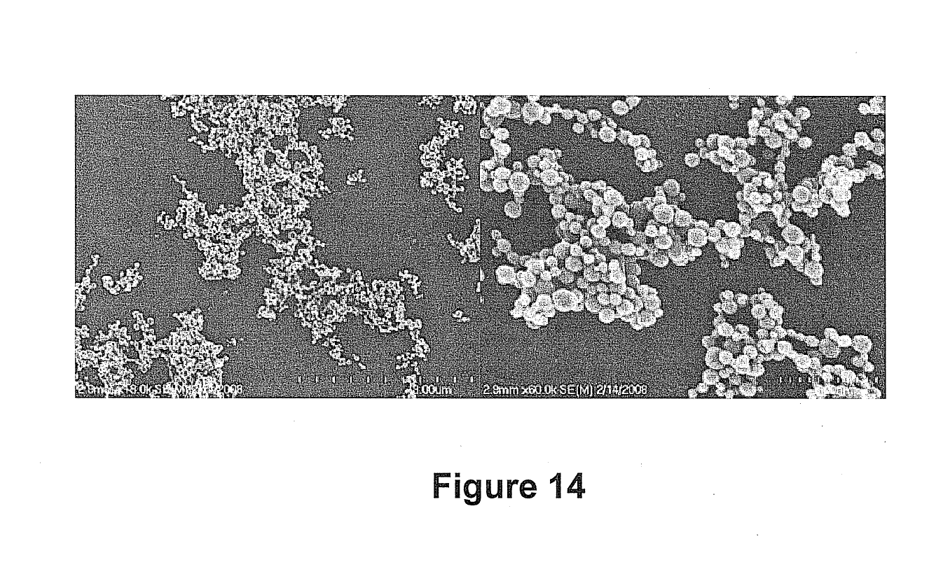

For example, the pH of a mixture of [c,c,t-Pt(NH₃)₂Cl₂(succ)₂].2N(CH₃)H₂ (2.5 μmmol) and ZnBr₂ (10.0 μmol) in 1.0 mL of distilled water was adjusted to pH 5.5 with dilute NaOH. Methanol (5.0 mL) was rapidly added to the magnetically stirred precursor solution, which resulted in the formation of a clear solution bluish-white in color. The resulting mixture was magnetically stirred in the dark for an additional 1 h. before isolating the product via centrifugation, washing with ethanol, and redispersing in ethanol via sonication. FIG. 14 shows the SEM micrographs of Znₓ[c,c,t-Pt(NH₃)₂Cl₂(O₂CCH₂CH₂CO₂)₂]_{y} nanoparticles synthesized via the rapid addition of methanol to a precursor (aqueous) solution of the components.

### Example 3

### Coordination Polymers with Ethylenediamine Tetraacetic acid or Succinate Bridging Ligands

### Example 3.1

### Synthesis of [c,c,t-Pt(NH₃)₂Cl₂(EDTA)]ₙ

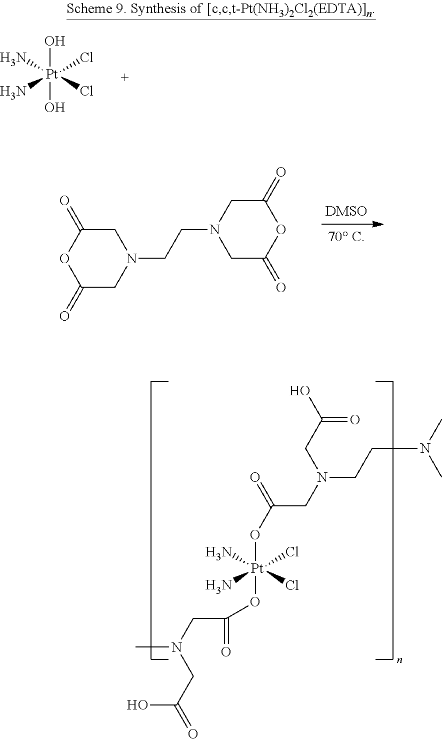

A mixture of c,c,t-Pt(NH₃)₂Cl₂(OH)₂ (50.0 mg, 0.150 mmol) and ethylenediamine tetraacetic acid (EDTA) dianhydride (38.4 mg, 0.150 mmol) in DMSO (anhydrous, 0.50 mL) was heated at 70° C. with magnetic stirring for 18 h, during which time the turbid pale yellow mixture became a solution. The solvent was removed under high vacuum at 70° C. to yield a yellow oily residue. The polymer was subsequently precipitated with ether, isolated, and dried in vacuo. The pale yellow solid was suspended in approximately 2 mL of distilled H₂O, and the pH of the suspension was adjusted to about 7 by titrating with dilute KOH. The resulting solution was dialyzed against water using 3500 molecular weight (MW) cutoff dialysis tubing.

### Example 3.2

### Synthesis of [c,c,t-Pt(NH₃)₂Cl₂(succ)]ₙ

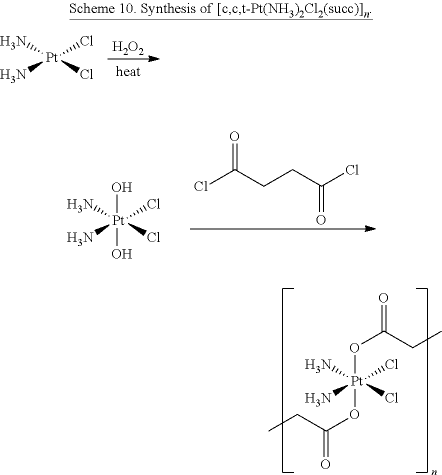

A mixture of c,c,t-Pt(NH₃)Cl₂(OH)₂ (50.0 mg, 0.150 mmol) and succinyl chloride (0.150 mmol) in 0.50 mL DMSO was heated at 70° C. with magnetic stirring for 18 h, during which time the turbid pale yellow mixture became a solution. The solvent was removed under high vacuum at 70° C. to yield a yellow oily residue. The polymer was subsequently precipitated with ether, isolated, and dried in vacuo. The pale yellow solid was suspended in approximately 2 mL of distilled H₂O, and the pH of the suspension was adjusted to approximately 7 by titrating with dilute KOH. The resulting solution was dialyzed against water using 3500 MW cutoff dialysis tubing.

### Example 3.3

### Assembly of Nanoparticles from Polymers Linked by Metal Ions

A precursor solution of the polymers described in Examples 3.2.1 or 3.2.2 and metal ion was prepared, and nanoparticles were subsequently precipitated via the addition of poor (initiator) solvent or using microemulsion methods as previously described.

### Example 4

### Surface Modification of Nanoscale Coordination Polymers (NCPs)

### Example 4.1

### Preparation of Polyvinylpyrolidone (PCP) Modified/Stabilized NCPs (NCP-1)

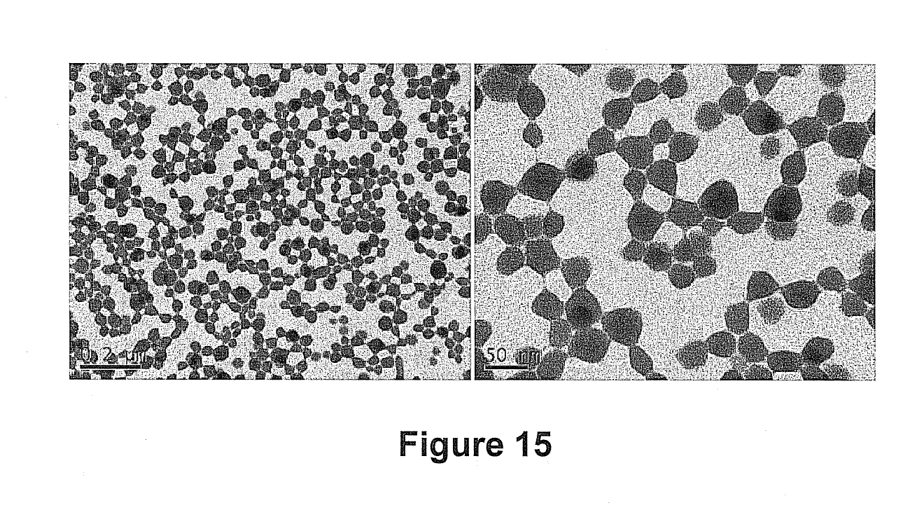

An ethanolic dispersion of NCP-1 was diluted to a final concentration of between about 2 and about 5 mg/mL with absolute ethanol. Approximately 0.02 mol equivalents of polyvinylpyrollidone (PVP, MW 40,000) were added to the dispersion, which was subsequently magnetically stirred for an additional 20-24 h. The PVP modified NCPs were isolated via centrifuge, washed with ethanol, and redispersed in ethanol. FIG. 15 shows the TEM micrographs of PVP-modified NCP-1 prepared according to the presently disclosed methods.

### Example 4.2

### Preparation of Silica-Modified NCP (NCP-1′)

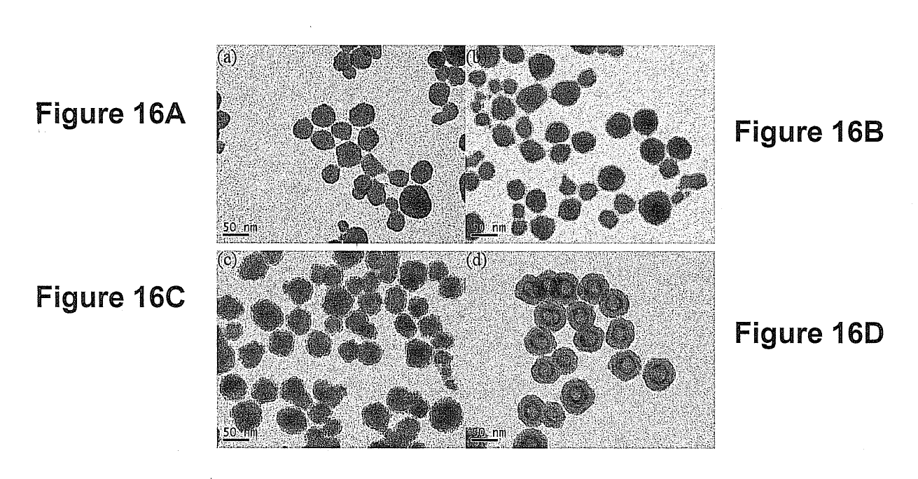

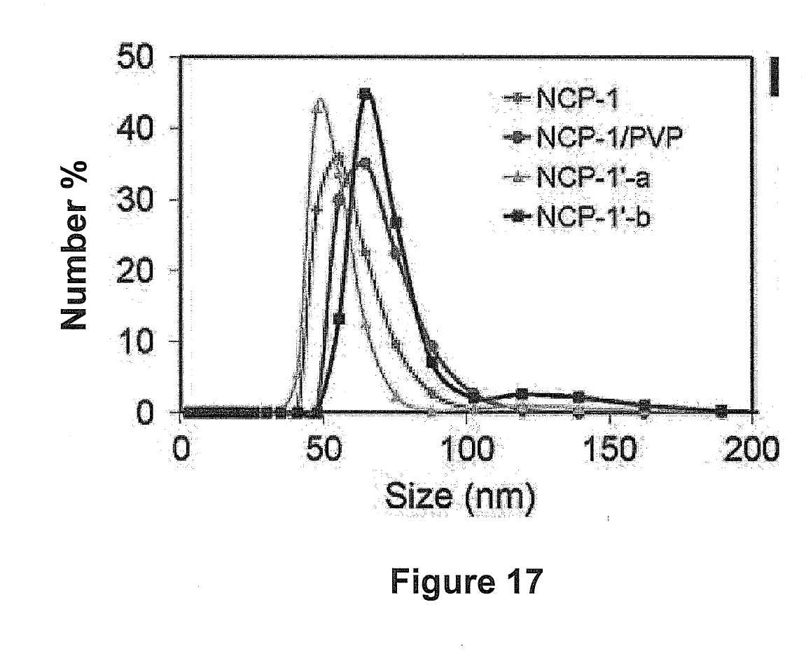

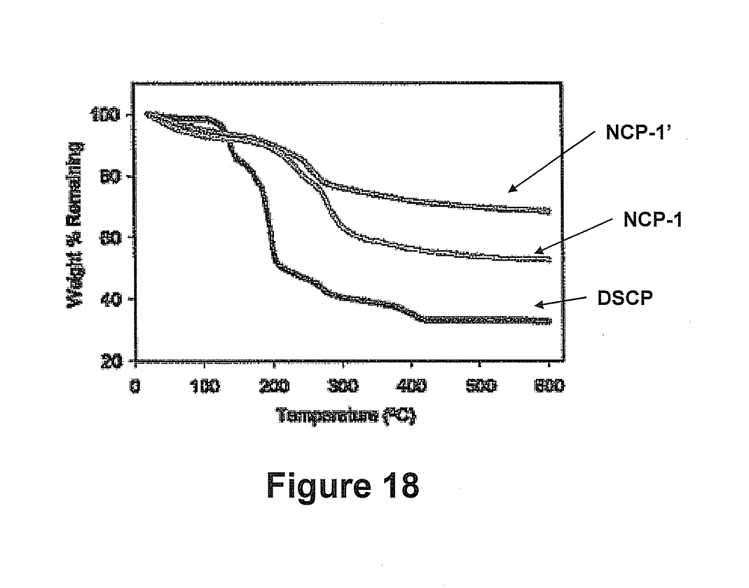

NCP-1 particles were coated with silica using sol-gel methodology. See Graf et al., Langmuir, 19, 6693-6700 (2003). An aliquot of the ethanolic dispersion of PVP-modified NCP-1 was diluted to a concentration of 0.2 mg/mL in 4% (v/v) NH₃ in ethanol. An aliquot of tetraethyl orthosilicate (TEOS, 2.5 μL/1.0 mg) was added to the reaction with magnetic stirring and the silica shell was allowed to evolve for at least 2 hours. Silica shell thickness typically increases with time and the volume of TEOS added to the reaction mixture. An additional aliquot of TEOS (5.0 μL/1.0 mg) can be added after 2 h if a thicker silica shell is desired. The SiO₂-modified NCPs were isolated via centrifuge, washed several times with ethanol, and redispersed in ethanol via sonication. FIGS. 14A-14D show the TEM micrographs of silica-coated NCP-1 isolated after 2 hours (i.e., NCP-1′-a) (FIG. 16A), three hours (FIG. 16B), four hours (i.e., NCP-1′-b) (FIG. 16C) and 7 hours (FIG. 16D) exposure to TEOS. For the nanoparticles shown in FIG. 16D, an additional aliquot of TEOS was added after the initial 2 hours. The NCP-1′-a particles had a silica layer thickness of about 2 nm (overall DLS diameter of 52.8±8.1 nm), while the NCP-1′-b particles had a silica layer thickness of about 7 nm (68.6±10.2 nm). See FIG. 17. FIG. 18 shows the TGA curve for the DSCP molecular complex, NCP-1 nanoparticles, and NCP-1′ particles. TGA gave a 7.0 and 8.5% reduction in the total weight loss for NCP-1′-a and NCP-1′-b, confirming the presence of the silica shell. The shell thickness was highly reproducible, varying only to a slight degree when the same reaction conditions were used on different samples.

### Example 4.3

### Dissolution Studies

About 3 mg of the NCPs dispersed in 2 mL of 2 mM HEPES buffer (pH 7.4 were dialyzed against 248 mL 2 mM HEPES buffer (pH 7.4) using 3500 MW cutoff cellulose dialysis tubing. Aliquots were removed from outside the dialysis bag to determine the moles of Pt released into the dialyzing buffer by ICP-MS. The percentage of the initial Pt dose released into the dialyzing buffer due to NCP dissolution was calculated using the equation:

% released={[Vₜₒₜ×C)+Y]/Z}×100

where Vₜₒₜ=total solvent volume remaining, C=Pt concentration as determined by ICP-MS, Y=total mole Pt removed from solution, and Z=Pt added to dialysis tubing.

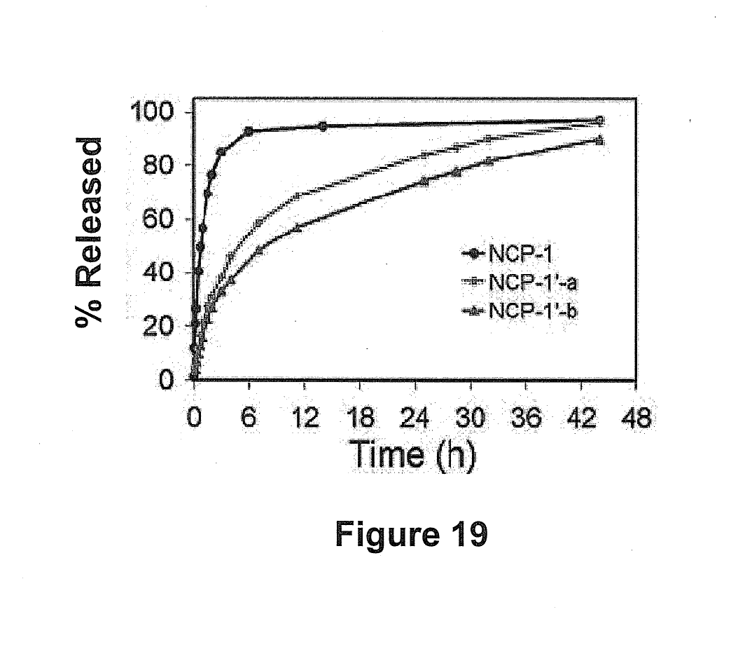

Pt species release varied according to silica layer thickness in the silica-coated NCPs, indicating that silica-stabilization of the NCPs can efficiently control the release of Pt species. The half-lives of dissolution for NCP-1′-a and NCP-1′-b were determined to be about 5.5 and about 9 hours, respectively. See FIG. 19. These rates should allow sufficient time for the Pt-based NCPs to circulate throughout the body and accumulate in tumor tissue. See Matsumura et al., Cancer Res., 46, 6387 (1986). Non-silica-coated NCPs (i.e., NCP-1) gave a half-life of dissolution of about 0.1 h.

### Example 5

### Synthesis of Targeted and Passivated NCPs

### Example 5.1

### Synthesis of Tri(ethoxysilylpropyl-c(RGDfK)

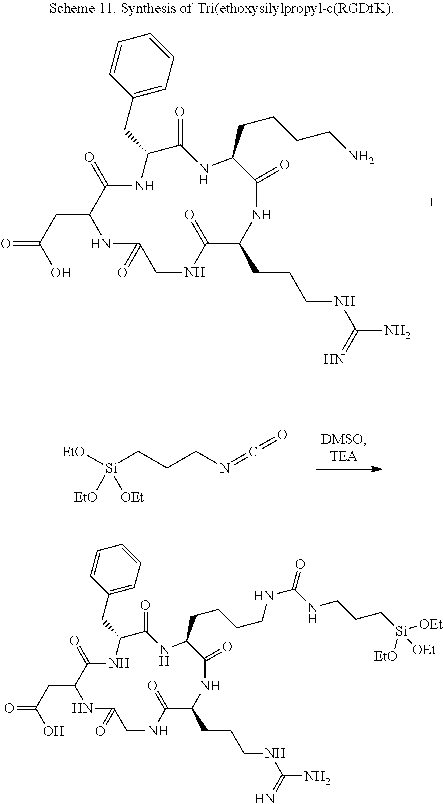

As shown in Scheme 11, cyclic RGDfK (c(RGDfK); 2.0 mg, 3.313 μmol) was added to a small round-bottom flask and dried under high vacuum for 1 hour. Anhydrous DMSO (500 μL) and triethylamine (0.20 pt) were added to the round-bottom flask, followed by 0.86 μL (3.44 μmol) of (3-isocyanatopropyl)triethoxysilane. The mixture was magnetically stirred under argon gas for 24 hours. The solution (4 mg c(RGDfK)/mL DMSO) was placed in a freezer for later use.

### Example 5.2

### Synthesis of Triethyoxysilylpropyl Carbamoyl-Poly(ethylene glycol) 2000

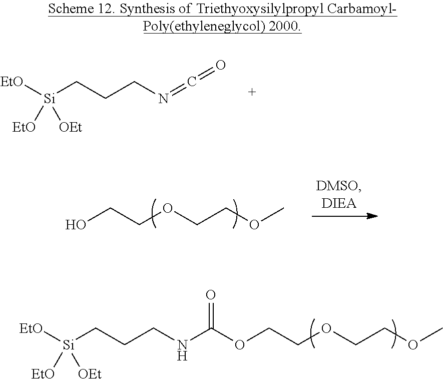

As shown in Scheme 12, 1.000 g (0.500 mmol) of poly(ethylene glycol)-2000-monomethyl ether was dried under vacuum at 100° C. for 1 hour. After cooling to room temperature, under nitrogen gas, the poly(ethylene glycol) was dissolved in 3 mL of anhydrous DMSO. 0.124 mL (0.124 g, 0.50 mmol) of distilled (3 isocyanatopropyl) triethoxysilane was then added, followed by 1 μL of Hünig base (0.74 mg, 0.0057 mmol, 1.15 mol %). The reaction was then stirred at room temperature, under nitrogen gas, for 24 hours. The DMSO was then removed under vacuum at 50° C. ¹H NMR (DMSO-d₆, 300 MHz): δ 0.51 (t, 2H), 1.14 (t, 9H), 1.43 (t, 2H), 2.92 (q, 2H), 3.35 (s, 3H), 3.50 (s, 128H), 3.73 (q, 6H), 4.03 (t, 2H), 7.22 (t, 1H).

### Example 5.3

### Functionalization of Silica-Modified NCPs with Silyl Derivatives

An aliquot of an ethanolic dispersion of silica-coated NCPs (e.g., NCP-1′) was diluted to a concentration of about 2 mg/mL of 4% NH⁴⁺OH⁻ (aq) in absolute ethanol. The desired weight % (up to 10% by mass) of the silyl-derivative molecule (e.g., the silyl derivative of a photosensitizer, radiosensitizer, passivating agent, imaging agent, or targeting agent) was added to the dispersion, and magnetic stirring was continued for an additional 20 h. The functionalized NCPs were isolated via centrifuge, washed with ethanol and DMSO, and dispersed in DMSO via sonication.

### Example 5.4

### Synthesis of c(RGDfK)-Functionalized NCP-1′

An aliquot of the c(RGDfK) DMSO solution (about 5 mass %) was added to a dispersion of NCP-1′ in 4% NH₃ in ethanol (approximately 2 mg/mL). The mixture was magnetically stirred at room temperature for 24 h. The c(RGDfK)-functionalized NCP-1′ particles were isolated via centrifugation, washed with ethanol and DMSO, and redispersed in DMSO via ultrasonication.

### Example 6

### In Vitro Activity of NCPs

Cell Lines All cell lines were purchased from the Tissue Culture Facility of the UNC Lineberger Comprehensive Cancer Center (Chapel Hill, N.C., United States of America) and cultured as per American Type Culture Collection (ATCC; Manassas, Va., United States of America) recommendations. HT29 cells (ATCC# HTB 38) were propagated in McCoy's 5A (Cellgro, Manassas, Va., United States of America) supplemented with 10% fetal bovine serum and 1% penicillin/streptomycin. MCF-7 cells (ATCC# HTB 22) were propagated in MEM Alpha (Cellgro, Manassas, Va., United States of America) supplemented with 10% fetal bovine serum, 1% penicillin/streptomycin, 1% sodium pyruvate, and 10 μg/mL insulin.

HT-29 viability assays: Angiogenic human colon cancer (HT-29) cells were grown in 96 well plates at 2000 cells/well to 100 μL total volume. After a 24 hour incubation, the media was replaced with 100 μL of drug solution containing 0.5% DMSO and 0.5% phosphate buffered saline (PBS) in media, with drug concentrations varying as indicated. All concentrations were performed in quadruplicate. Cell viability was measured after 72 hours using CellTiter 96 Aqueous One Solution Assay (Promega Corporation, Madison, Wis., United States of America), according to the manufacturer's protocol.

Treatment of HT-29 cells with DSCP, NCP-1 and NCP-1′ did not lead to any appreciable cell death, presumably because the DSCP species released from NCP-1 and NCP-1′ do not have a pathway to enter the cells effectively. Further, there were no reductants in the media under the in vitro conditions used to transform DSCP into the active Pt(II) species. The released DSCP would become active in vivo through reduction with endogenous biomolecules, such as glutathione.

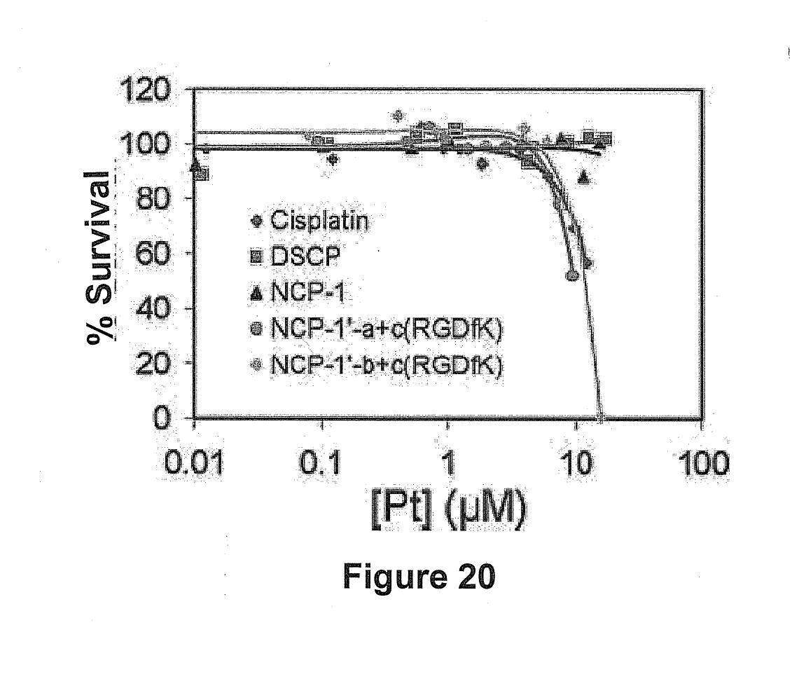

To enhance the cellular uptake of NCPs in vitro, silyl-derived cyclic(RGDfK) was grafted onto the surfaces of the nanoparticles as described in Example 5. Cyclic(RGDfk) is a small cyclic peptide sequence exhibiting high binding for the αᵥβ₃ integrin upregulated in many angiogenic cancers. Cyclic(RGDfK)-targeted NCP-1′-a and NCP-1′-b gave IC₅₀ values of 9.7 and 11.9 μM respectively. See FIG. 20. These results indicate that the targeted NCPs are sufficiently internalized by the cells, for example, by receptor-mediated endocytosis. Inside the cells, the DSCP species can be released from the silica-coated NCPs and reduced to the active Pt(II) species by intracellular reductants.

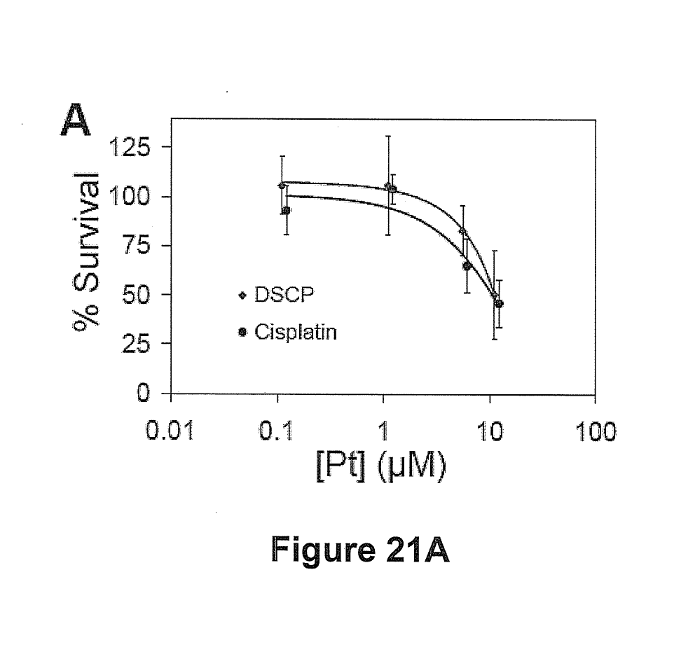

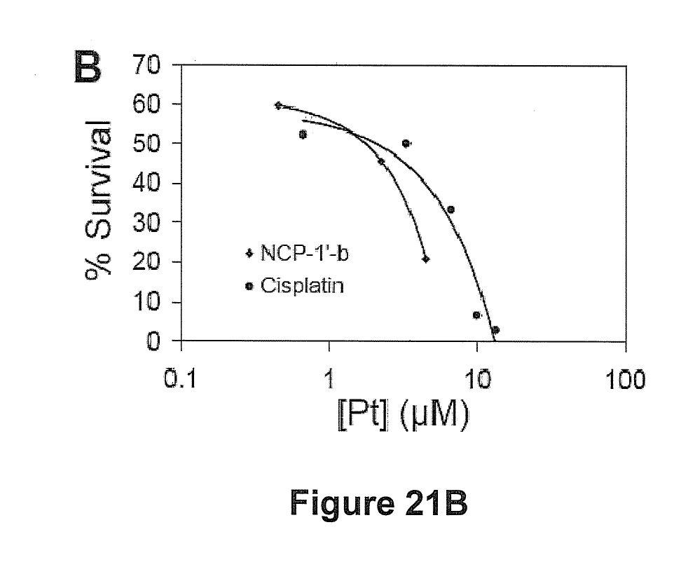

MCF-7 Viability Assays. As a further control, cell viability assays were also performed using MCF-7 cells (breast cancer). MCF-7 cells were assayed under the same conditions as HT-29, and also at 4000 cells/well and 72 hours. Unlike with HT-29, Pt(IV) drugs are active. As shown in FIG. 21A, DSCP is roughly equivalent to cisplatin in activity at 4000 cells/well, with an IC₅₀ of 11.1 μM for DSCP versus 10.7 μM for cisplatin. NCP-1′-b was also tested but at 2000 cells/well. It gave an IC₅₀ value of 1.8 μM versus 2.1 μM for cisplatin, as shown in FIG. 21B. Unlike with HT-29, MCF-7 does not overexpress the αᵥβ₃ integrin, and so c(RGDfK) targeting was not necessary.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A nanoparticle comprising a coordination polymer comprising a plurality of platinum metal complexes.

2. The nanoparticle of claim 1, wherein the plurality of platinum metal complexes consist of a plurality of platinum (II) metal complexes, a plurality of platinum (IV) metal complexes, or a combination thereof.

3. The nanoparticle of claim 1, wherein one or more of the platinum metal complexes comprises:
a platinum metal atom;
at least one nonbridging ligand, wherein the at least one nonbridging ligand is bonded to the platinum metal atom through at least one coordination bond; and
at least one bridging ligand, wherein the at least one bridging ligand is bonded to the platinum metal atom through at least one coordination bond and comprises at least one linking moiety, wherein each of the at least one linking moiety is bonded to an additional metal atom via a coordination bond.

4. The nanoparticle of claim 3, wherein each of the at least one linking moiety is independently selected from the group consisting of a carboxylate, a carboxylic acid, an amine, a hydroxyl, a thiol, a carbamate, an ester, a phosphate, a phosphonate, a carbonate, and an amide.

5. The nanoparticle of claim 3, wherein each of the at least one bridging ligand is independently selected from the group consisting of a polymeric bridging ligand and a nonpolymeric bridging ligand.

6. The nanoparticle of claim 5, wherein each of the at least one bridging ligand is a nonpolymeric bridging ligand.

7. The nanoparticle of claim 6, wherein the bridging ligand comprises at least two carboxylate groups.

8. The nanoparticle of claim 6, wherein at least one platinum metal complex comprises two bridging ligands, wherein each of the two bridging ligands is bonded to the platinum metal atom through one coordination bond and comprises at least one linking moiety.

9. The nanoparticle of claim 8, wherein each of the two bridging ligands is independently selected from the group consisting of 1,4-benzene dicarboxylate; 1,3,5-benzene tricarboxylate; succinate; and ethylene diamine tetraacetate.

10. The nanoparticle of claim 6, wherein at least one platinum metal complex comprises one bridging ligand, wherein the one bridging ligand is bonded to the platinum metal atom through two coordination bonds and comprises at least two linking moieties.

11. The nanoparticle of claim 10, wherein the one bridging ligand is a bipyridine dicarboxylate.

12. The nanoparticle of claim 11, wherein the bipyridine dicarboxylate is selected from 2,2′-bipyridine-5,5′-dicarboxylate and 2,2′-bipyridine-4,4′-dicarboxylate.

13. The nanoparticle of claim 6, wherein one of the at least one bridging ligand is a nonplatinum anticancer drug.

14. The nanoparticle of claim 13, wherein the nonplatinum anticancer drug is selected from the group consisting of methotrexate, folic acid, leucovorin, vinblastine, vincristine, melphalan, pemetrexed, vindesine, anastrozole, doxorubicin, cytarabine, azathioprine, letrozole and carboxylates thereof.

15. The nanoparticle of claim 5, wherein one of the at least one bridging ligand is a polymeric bridging ligand.

16. The nanoparticle of claim 15, wherein the polymeric bridging ligand comprises one of the group consisting of poly(silsesquioxane), poly(siloxane), poly(acrylate) and poly(acrylamide).

17. The nanoparticle of claim 3, wherein the additional metal atom is a platinum metal atom of a second platinum metal complex.

18. The nanoparticle of claim 3, wherein the additional metal atom is a nonplatinum metal atom selected from the group consisting of a transition metal atom, a lanthanide metal atom, and an actinide metal atom.

19. The nanoparticle of claim 18, wherein the additional metal atom is selected from the group consisting of Tb³⁺ and Zn²⁺.

20. The nanoparticle of claim 3, wherein each of the at least one nonbridging ligands is independently selected from the group consisting of NH₃, a primary amine, a secondary amine, a diamine, an aromatic amine, a halide, and hydroxide.

21. The nanoparticle of claim 20, wherein the diamine is a cyclohexanediamine.

22. The nanoparticle of claim 20, wherein each of the at least one nonbridging ligands is independently selected from the group consisting of NH₃ and chloride.

23. The nanoparticle of claim 1, wherein each of the plurality of platinum metal complexes is independently selected from the group consisting of:
Pt[(NH₃)₂(Cl)₂(O₂CCH₂CH₂CO₂)₂];
Pt[(NH₃)₂(Cl)₂{O₂CC₆H₃(CO₂)₂}₂], dichloro(2,2′-bipyridine-4,4′-dicarboxylato)platinum (II);
dichloro(2,2′-bipyridine-5,5′-dicarboxylato)platinum (II), and
Pt[(NH₃)₂(Cl)₂(ethylene diamine tetraacetate)₂].

24. The nanoparticle of claim 1, wherein the nanoparticle has a diameter ranging between about 20 nm and about 250 nm.

25. The nanoparticle of claim 24, wherein the nanoparticle has a diameter ranging between about 40 nm and about 70 nm.

26. The nanoparticle of claim 1, further comprising one or more of the group consisting of a photosensitizer, a radiosensitizer, a radionuclide, an imaging agent, and a targeting agent.

27. The nanoparticle of claim 26, wherein the imaging agent is selected from the group consisting of an optical imaging agent, a magnetic resonance imaging (MRI) agent, a positron emission tomography (PET) imaging agent, and a single photon emission computed tomography (SPECT) imaging agent.

28. The nanoparticle of claim 27, wherein the optical imaging agent is a luminescent agent.

29. The nanoparticle of claim 26, wherein the targeting agent is selected from the group consisting of a small molecule, a peptide, and a protein.

30. The nanoparticle of claim 29, wherein the targeting agent binds to a receptor or ligand present on a cancer cell.

31. The nanoparticle of claim 30, wherein the targeting agent comprises cyclic(RGDfk).

32. The nanoparticle of claim 1, wherein an outer surface of the nanoparticle is chemically modified with one or more of the group consisting of a passivating agent, a targeting agent, and an imaging agent.

33. The nanoparticle of claim 32, wherein the passivating agent comprises poly(ethylene glycol).

34. The nanoparticle of claim 1, comprising a core and an outer layer, the core comprising a coordination polymer comprising a plurality of platinum metal complexes, and the outer layer surrounding the core and comprising one of the group consisting of a metal oxide, a lipid bilayer, an organic polymer, a silica-based polymer, and combinations thereof.

35. The nanoparticle of claim 34, wherein the organic polymer is polyvinylpyrolidone (PVP).

36. The nanoparticle of claim 34, wherein the outer layer is polyvinylpyrolidone (PVP), SiO₂, or a combination thereof.

37. The nanoparticle of claim 34, wherein the outer layer has a thickness ranging between about 1 nm and about 10 nm.

38. A pharmaceutical composition comprising a nanoparticle of claim 1 and a pharmaceutically acceptable carrier.

39. The pharmaceutical composition of claim 38, wherein the pharmaceutical composition is pharmaceutically acceptable in humans.

40. The pharmaceutical composition of claim 38, wherein the pharmaceutical composition comprises one of a liposome and a microemulsion.

41. A method of inhibiting proliferation of a cell, the method comprising contacting the cell with a nanoparticle, wherein the nanoparticle comprises a coordination polymer comprising a plurality of platinum metal complexes.

42. The method of claim 41, wherein the nanoparticle further comprises one or more of the group consisting of a photosensitizer, a radiosensitizer, a radionuclide, a passivating agent, an imaging agent, and a targeting agent.

43. The method of claim 41, wherein the coordination polymer comprises one or more nonplatinum anticancer drugs, wherein each of the one or more nonplatinum anticancer drugs is selected from the group consisting of methotrexate, folic acid, leucovorin, vinblastine, vincristine, melphalan, imatinib, pemetrexed, vindesine, anastrozole, doxorubicin, cytarabine, azathioprine, letrozole and carboxylates thereof.

44. The method of claim 41, wherein the coordination polymer further comprises one or more nonplatinum metal atom selected from the group consisting of a transition metal atom, a lanthanide metal atom, and an actinide metal atom.

45. The method of claim 41, wherein the nanoparticle comprises a core and an outer layer, the core comprising a coordination polymer comprising a plurality of platinum metal complexes, and the outer layer surrounding the core and comprising one of the group consisting of a metal oxide, a lipid bilayer, an organic polymer, a silica-based polymer, and combinations thereof.

46. The method of claim 41, wherein each of the plurality of platinum metal complexes is selected from the group consisting of:
Pt[(NH₃)₂(Cl)₂(O₂CCH₂CH₂CO₂)₂];
Pt[(NH₃)₂(Cl)₂{O₂CC₆H₃(CO₂)₂}₂];
dichloro(2,2′-bipyridine-4,4′-dicarboxylato)platinum (II);
dichloro(2,2′-bipyridine-5,5′-dicarboxylato)platinum (II); and
Pt[(NH₃)₂(Cl)₂(ethylene diamine tetraacetate)₂].

47. The method of claim 41, wherein the cell is a cancer cell, optionally selected from the group consisting of a skin cancer cell, a connective tissue cancer cell, an esophageal cancer cell, a head and neck cancer cell, a breast cancer cell, a lung cancer cell, a stomach cancer cell, a pancreatic cancer cell, an ovarian cancer cell, a cervical cancer cell, a uterine cancer cell, an anogenital cancer cell, a kidney cancer cell, a bladder cancer cell, a colon cancer cell, a prostate cancer cell, a retinal cancer cell, a central nervous system cancer cell, and a lymphoid cancer cell.

48. The method of claim 47, wherein the cancer cell is selected from the group consisting of a breast cancer cell and a colon cancer cell.

49. A method of treating cancer in a subject in need of treatment thereof, the method comprising administering to the subject a nanoparticle comprising a coordination polymer, wherein the coordination polymer comprises a plurality of platinum metal complexes.

50. The method of claim 49, wherein the coordination polymer comprises one or more nonplatinum anticancer drugs, wherein each of the one or more nonplatinum anticancer drugs is selected from the group consisting of methotrexate, folic acid, leucovorin, vinblastine, vincristine, melphalan, imatinib, pemetrexed, vindesine, anastrozole, doxorubicin, cytarabine, azathioprine, letrozole and carboxylates thereof.

51. The method of claim 49, wherein the coordination polymer further comprises one or more nonplatinum metal atom selected from the group consisting of a transition metal atom, a lanthanide metal atom, and an actinide metal atom.

52. The method of claim 49, wherein the nanoparticle comprises a core and an outer layer, the core comprising a coordination polymer comprising a plurality of platinum metal complexes, and the outer layer surrounding the core and comprising one of the group consisting of a metal oxide, a lipid bilayer, an organic polymer, a silica-based polymer, and combinations thereof.

53. The method of claim 52, wherein the outer later comprises polyvinylpyrrolidinone (PVP), SiO₂, or a combination thereof.

54. The method of claim 49, wherein each of the plurality of platinum metal complexes is selected from the group consisting of:
Pt[(NH₃)₂(Cl)₂(O₂CCH₂CH₂CO₂)₂];
Pt[(NH₃)₂(Cl)₂{O₂CC₆H₃(CO₂)₂}₂];
dichloro(2,2′-bipyridine-4,4′-dicarboxylato)platinum (II);
dichloro(2,2′-bipyridine-5,5′-dicarboxylato)platinum (II); and
Pt[(NH₃)₂(Cl)₂(ethylene diamine tetraacetate)₂].

55. The method of claim 49, wherein the cancer is selected from a skin cancer, a connective tissue cancer, an esophageal cancer, a head and neck cancer, a breast cancer, a lung cancer, a stomach cancer, a pancreatic cancer, an ovarian cancer, a cervical cancer, a uterine cancer, an anogenital cancer, a kidney cancer, a bladder cancer, a colon cancer, a prostate cancer, a retinal cancer, a central nervous system cancer, and a lymphoid cancer.

56. The method of claim 55, wherein the cancer is selected from the group consisting of breast cancer and colon cancer.

57. The method of claim 49, wherein the nanoparticle further comprises one or more of the group consisting of a photosensitizer, a radiosensistizer, a radionuclide, a passivating agent, an imaging agent, and a targeting agent.

58. The method of claim 57, further comprising imaging delivery of the nanoparticle in one or more tissue or organ in the subject following administration of the nanoparticle.

59. The method of claim 57, further comprising administering to the subject an external stimulus selected from the group consisting of laser light and X-ray radiation.

60. The method of claim 49, wherein an outer surface of the nanoparticle is chemically modified with one or more of the group consisting of a passivating agent, a targeting agent, and an imaging agent.

61. The method of claim 49, wherein the nanoparticle is administered to the subject in a liposome or a microemulsion.

62. The method of claim 49, wherein the subject is a mammal.

63. A method of synthesizing a nanoparticle comprising a coordination polymer comprising a plurality of platinum metal complexes, wherein the method comprises precipitation or use of a microemulsion system.

64. The method of claim 63, wherein the method comprises:
providing a solution comprising a first solvent, at least one bridging ligand precursor, and a plurality of platinum diaqua complexes selected from the group consisting of platinum (II) diaqua complexes, platinum (IV) diaqua complexes, and mixtures thereof; and
adding a second solvent to the solution to precipitate the nanoparticle.

65. The method of claim 64, further comprising adjusting the pH of the solution prior to adding the second solvent.

66. The method of claim 64, wherein the first solvent comprises water, dimethyl sulfoxide (DMSO), or a combination thereof.

67. The method of claim 64, wherein the at least one bridging ligand precursor is selected from the group consisting of a benzene dicarboxylic acid, a benzene dicarboxylate, a carboxylate-substituted styrene, a carboxylate-substituted silyl ether, a bipyridine dicarboxylic acid, a bipyridine dicarboxylate, a dicarboxylic anhydride, a diacyl dichloride, and a nonplatinum anticancer drug.

68. The method of claim 67, wherein the nonplatinum anticancer drug is selected from the group consisting of methotrexate, folic acid, leucovorin, vinblastine, vincristine, melphalan, imatinib, pemetrexed, vindesine, anastrozole, doxorubicin, cytarabine, azathioprine, letrozole, and carboxylates thereof.

69. The method of claim 64, wherein the second solvent is selected from the group consisting of acetone, an alcohol, ether, and acetonitrile.

70. The method of claim 64, wherein the solution further comprises a metal complex comprising a nonplatinum metal atom selected from the group consisting of a transition metal atom, a lanthanide metal atom, and an actinide metal atom.

71. The method of claim 70, wherein the nonplatinum metal atom is selected from Tb³⁺ and Zn²⁺.

72. The method of claim 64, wherein the solution further comprises a polymerizable monomer.

73. The method of claim 72, wherein the polymerizable monomer is selected from the group consisting of acrylic acid, acrylamide, and a silyl ether.

74. The method of claim 64, wherein the solution further comprises an additional component, wherein the additional component is selected from the group consisting of a radionuclide, an imaging agent, a photosensitizer, and a radiosensitizer, and adding the second solvent co-precipitates the additional component, thereby incorporating the additional component into the nanoparticle.

75. The method of claim 63, wherein the method comprises:
providing a first mixture comprising a microemulsion system comprising water, an organic solvent, a surfactant, and a co-surfactant;
adding to the first mixture an aqueous solution comprising a platinum metal complex, thereby forming a second mixture, wherein the platinum metal complex comprises a platinum metal atom, one or more nonbridging ligands, and at least one ligand bound to the platinum metal atom by at least one coordination bond and comprising at least one prelinking moiety, wherein the at least one prelinking moiety is a group that can form a coordination bond with an additional metal atom;
stirring the second mixture until the second mixture is visably clear;
providing a third mixture comprising a microemulsion system comprising water, an organic solvent, a surfactant, and a co-surfactant;
adding to the third mixture an aqueous solution comprising a nonplatinum metal compound, thereby forming a fourth mixture;
stirring the fourth mixture until the fourth mixture is visably clear;
adding the fourth mixture and the second mixture to form a fifth mixture; and
stirring the fifth mixture for a period of time, thereby synthesizing the nanoparticle.

76. The method of claim 75, wherein the nonplatinum metal compound comprises a nonplatinum metal atom selected from the group consisting of a transition metal atom, a lanthanide metal atom, and an actinide metal atom.

77. The method of claim 76, wherein the nonplatinum metal compound is TbCl₃.

78. The method of claim 63, wherein the method comprises:
providing a microemulsion system comprising water; an organic solvent; a surfactant; a co-surfactant; a polymerizable monomer; and a platinum metal complex, wherein the platinum metal complex comprises a platinum metal atom, one or more nonbridging ligands, and at least one ligand bonded to the platinum metal atom by at least one coordination bond and comprising at least one prelinking moiety, where the at least one prelinking moiety is a moiety that can react with the polymerizable monomer.

79. The method of claim 78, wherein the prelinking moiety is selected from the group consisting of an alkyl halide, an acyl halide, a silyl ether, an alkene, an alkyne, a carboxylic acid, an amine, an ester, an anhydride, and an isocyanate.

80. The method of claim 78, wherein the polymerizable monomer is selected from the group consisting of a silyl ether, acrylic acid, and acrylamide.

81. The method of claim 63, further comprising isolating the nanoparticle via centrifugation.

82. The method of claim 63, further comprising coating the nanoparticle with one or more of the group consisting of a metal oxide, a lipid bilayer, an organic polymer, a silica-based polymer, and combinations thereof.

83. The method of claim 63, further comprising grafting onto the surface of the nanoparticle one or more of a photosensitizer, a radiosensitizer, a radionuclide, an imaging agent, a passivating agent, and a targeting agent.

84. A coordination polymer comprising a plurality of platinum metal complexes wherein the platinum metal complexes are linked via bridging ligands, wherein each bridging ligand is independently selected from the group consisting of a nonpolymeric bridging ligand and a polymeric bridging ligand.

85. The coordination polymer of claim 84, wherein the nonpolymeric bridging ligand is a nonplatinum anticancer drug selected from the group consisting of methotrexate, folic acid, leucovorin, vinblastine, vincristine, melphalan, vincristine, imatinib, pemetrexed, vindesine, anastrozole, doxorubicin, cytarabine, azathioprine, letrozole, and carboxylates thereof.

86. The coordination polymer of claim 84, wherein the polymeric bridging ligand is selected from the group consisting of poly(silsesquioxane), poly(siloxane), poly(acrylate) and poly(acrylamide).

87. The coordination polymer of claim 84, wherein each of the plurality of platinum metal complexes is independently selected from the group consisting of Pt[(NH₃)₂(Cl)₂(O₂CCH₂CH₂CO₂)₂], Pt[(NH₃)₂(Cl)₂{O₂CC₆H₃(CO₂)₂}₂]; dichloro(2,2′-bipyridine-4,4′-dicarboxylato)platinum (II); dichloro(2,2′-bipyridine-5,5′-dicarboxylato)platinum (II); and Pt[(NH₃)₂(Cl)₂(ethylene diamine tetraacetate)₂].

88. The coordination polymer of claim 84, further comprising a plurality of nonplatinum metal atoms, wherein the nonplatinum metal atoms are each independently selected from the group consisting of a transition metal atom, a lanthanide metal atom, and an actinide metal atom.

89. The coordination polymer of claim 88, wherein the nonplatinum metal atom is selected from Tb³⁺, Zn²⁺, and combinations thereof.

90. A coordination polymer comprising a plurality of nonplatinum metal complexes wherein the nonplatinum metal complexes are linked via bridging ligands, wherein one or more of the bridging ligands is a nonplatinum anticancer drug.

91. A nanoparticle comprising the coordination polymer of claim 90.

92. The nanoparticle of claim 91, further comprising one or more of the group consisting of a photosensitizer, a radiosensitizer, a radionuclide, an imaging agent, a passivating agent, a stabilizing agent, and a targeting agent.
